SEQUENCE LISTING (3 sequences)

SEQ ID NO: 1 - DNA, 21 bp, synthetic construct
  source 1..21: mol_type = other DNA
agccacaccatcctagttgcc

SEQ ID NO: 2 - DNA, 18 bp, synthetic construct
  source 1..18: mol_type = other DNA
ggaagacagaatggaaga

SEQ ID NO: 3 - DNA, 19 bp, synthetic construct
  source 1..19: mol_type = other DNA
caaggttaccatataccca

(19) United States
(12) Patent Application Publication
(10) Pub. No.: US 2026/0125765 A1
(43) Pub. Date: May 7, 2026

(54) KIDNEY CANCER SCREENING METHODS

(71) Applicant: Cicadea Biotech, LLC, St. Louis, MO (US)

(72) Inventor: Wen Chen Burns, Chesterfield, MO (US)

(21) Appl. No.: 19/429,755

(22) Filed: Dec. 22, 2025

Related U.S. Application Data
(62) Division of application No. 18/449,442, filed on Aug. 14, 2023.
(60) Provisional application No. 63/397,618, filed on Aug. 12, 2022.

(51) Int. Cl. C12Q 1/6886; C12N 15/10

(52) U.S. Cl. CPC C12Q 1/6886; C12N 15/1017

(57) ABSTRACT

Device and methods for detection of kidney cancer using urine samples are disclosed herein. The device and methods enable isolation and quantification of kidney tumor cells in urine samples from a subject in need thereof.

### CROSS-REFERENCE TO RELATED APPLICATIONS

The present application is a divisional of U.S. patent application Ser. No. 18/449,442, filed Aug. 14, 2023, which claims the benefit of U.S. Provisional Patent Application No. 63/397,618, filed Aug. 12, 2022, the contents of each of which is hereby incorporated by reference in its entirety.

### SEQUENCE LISTING

The instant application contains a Sequence Listing which has been submitted electronically in XML format and is hereby incorporated by reference in its entirety for all purposes. The XML copy, created on Dec. 19, 2025, is referred to as 111315-865714_Sequence_Listing.xml and is 3,682 bytes in size.

### FIELD

The present disclosure provides methods, devices, and kits for diagnosing kidney cancer from urine samples in a subject in need thereof.

### BACKGROUND

The American Cancer Society's estimates that about 81,800 new cases of kidney cancer (52,360 in men and 29,440 in women) will be diagnosed in the US for 2023 and about 14,890 people (9,920 men and 4,970 women) will die from this disease each year. Renal cell carcinomas (RCC) are the most common form of kidney cancer, however other forms of kidney cancers include transitional cell carcinomas, Wilms tumors, and renal sarcomas. Renal cell carcinoma is found mostly in high-risk groups of patients over the age of 65 in US. An estimated 18.2% of renal cancer cases are fatal. Most early-stage renal cancer patients do not present with symptoms and are incidentally found during a CT-scan for other purposes. If treated when the disease is localized to the kidney (stage 1-2), 5-year survival rates for renal cancer are 93%. However, the survival rate drops significantly, to as low as 15%, with locally advanced or metastatic disease. Even if treated, renal cancer may have a recurrence rate of 20% to 40% after removal of a kidney (nephrectomy).

Early diagnosis is therefore key to favorable outcomes for a subject suffering from kidney cancer. However, currently methods for detecting kidney cancer are limited in ability to detect the cancer early. Detection also often occurs after the cancer has already metastasized to other locations and the patient is experiencing symptoms. These diagnoses preferentially use CT Scan imaging, which exposes the patient to harmful radiation. Moreover, such testing is costly which deters referring a patient for screening unless the patient presents with specific symptoms (such as blood in urine, unexplained flank pain, cough, bone pain, or other symptoms of advanced or metastatic disease) that are deemed to warrant CT scan analysis. Current methods are not sufficiently sensitive for early detection of kidney cancer from urine samples. Additionally, blood testing for genetic markers is limited by the fact that no single biomarker is validated for rapid screening of all mutations of kidney cancer genes. An urgent need exists for a simple, non-invasive, effective screening method for early detection of primary and recurrent renal cancer in the urinary tract.

### SUMMARY

In some aspects the current disclosure encompasses a method for screening for a target analyte in a urine sample from a subject in need thereof, the method comprising the steps of: obtaining or having obtained the urine sample; passing the urine sample through a filtration member to obtain a retentate, wherein the retentate is enriched in urine tumor cells (UTCs), and detecting the target analyte present in the retentate. In some aspects, the subject is at risk of having, suspected of having, or has a current or prior diagnosis of kidney cancer. In some aspects, the method further comprises quantifying the target analyte. In some aspects, the method of quantification comprises quantitative Polymerase chain reaction (qPCR). In some aspects, the target analyte comprises a discerning sequence or a housekeeping gene or a fragment thereof. In some aspects, the housekeeping gene is GAPDH. In some aspects, the quantification of target analyte is used to estimate the tumor cell density in urine. In some aspects, the tumor cells are kidney cancer cells, bladder cancer cells, urinary tract cancer cells, prostate cancer cells, or testicle cancer cells. In some aspects, the filtration member comprises a membrane with a pore size of about 8 microns.

In further aspects, provided herein is a method for classifying a subject suspected or at risk of having kidney cancer, comprising:
(a) obtaining or having obtained a urine sample from the subject;
(b) passing the urine sample through a filtration member to obtain a retentate, wherein the retentate is enriched in kidney tumor cells;
(c) lysing or disrupting the intact cells in the retentate to form a lysed/disrupted cell mixture comprising nucleic acid from lysed/disrupted cells;
(d) filtering the lysed/disrupted cell mixture from (c) through the filtration membrane to form a filtrate comprising the nucleic acid from the lysed/disrupted cells;
(e) subjecting the filtrate from (d) to a quantitative polymerase chain reaction (qPCR) to quantify the level of a target analyte present in the retentate;
(f) comparing the level of the target analyte in the urine sample to a reference level of the target analyte in a control sample;
(g) determining the tumor cell density in urine in the retentate;
(h) detecting the presence of kidney cancer in the subject if the level of the target analyte or the tumor cell density in the urine sample is elevated compared to the reference level; and
(i) classifying the subject as a candidate for a kidney cancer therapy based on step (h).

In some aspects, the method of (c) or (d) further comprises isolating the DNA from the kidney tumor cells in the retentate, and conducting a qPCR analysis using primers and probe targeting a discerning DNA sequence, sequences, or a fragment thereof.

In some aspects of the method, the target analyte comprises a housekeeping gene or a fragment thereof. In some aspects, the housekeeping gene is GAPDH. In some aspects, the filtration member comprises a membrane with a pore size of about 8 microns.

In further aspects, the disclosure further encompasses a device for screening for kidney cancer cells in a urine sample, the device comprising:
a. a container member having a bottom surface having a center point;
b. a first filtration member having a center point, the first filtration member concentrically positioned adjacent to the container member.

In some aspects, the first filtration member comprises an ultra-high molecular weight polyethylene with a pore size of about 8 microns. In some aspects, the device is adapted to be used with a centrifuge or vacuum. In some aspects, the device of further comprises a cap member, a washer, a lid, or any combination thereof.

In some aspects, further provided herein is a method for detecting kidney cancer in a subject at risk or suspected of having kidney cancer, comprising the steps of:
(a) obtaining or having obtained the urine sample from the subject;
(b) passing the urine sample through a filtration member comprising a filtration membrane having a pore size of about 8 microns to obtain a retentate, wherein the retentate is enriched in kidney cancer or tumor cells;
(c) lysing or disrupting the intact cells in the retentate to form a lysed/disrupted cell mixture comprising nucleic acid from lysed/disrupted cells;
(d) filtering the lysed/disrupted cell mixture from (c) through the filter to form a filtrate comprising the nucleic acid from the lysed/disrupted cells;
(e) subjecting the filtrate from (d) to a polymerase chain reaction (PCR) to quantify the nucleic acid of a target analyte;
(f) determining the tumor cell density in the retentate.

In some aspects of the method for detecting kidney cancer, the target analyte is GAPDH DNA or a fragment thereof. In some aspects the target analyte comprises a mammalian housekeeping gene such as GAPDH DNA or a fragment thereof. In some aspects the filtration member comprises a membrane with a cut-off of about 8 microns.

In some aspects the subject is a mammal. In some aspects the subject is a human.

### BRIEF DESCRIPTION OF THE FIGURES

FIG. 1 depicts a schematic overview of the device to isolate kidney tumor cells from urine samples (UTC). The device comprises a container member (100), a filtration member (200), a membrane associated with the filtration member (300) and a lid (400). The filtration member may be removable (see figure on the right) and may be closed with a cap (500).

FIG. 2A-2D depicts exemplary devices and methods of using the devices.

FIG. 2A depicts exemplary configurations of the device contemplated. FIG. 2B-2D schematically depicts use of the device for urine-analysis. Further shown is DNA isolation from tumor cells isolated on the filtration member.

FIG. 3 is a bar graph showing the estimated number of UTC in urine sample before, after 2 weeks and after 2 months of nephrectomy from a kidney cancer patient.

FIG. 4 is a line graph showing log copy number of tumor cells vs. tumor size in cm. from urine samples analyzed from group of kidney cancer patients.

FIG. 5 shows comparison of log number of UTC in urine over months after surgery from a kidney cancer patient.

### DETAILED DESCRIPTION

One of the main challenges in detecting biomarkers for diagnosis and grading of kidney cancer using urinalysis is to obtain sufficient tumor cells (e.g., UTC) for downstream analysis. This becomes even more challenging in early stages of cancer. The current-state-of-the-art uses microscopic analysis which results in a very high false-negative rate as the analysis is dependent of the field of view. In some instances, the samples can be centrifuged to concentrate cells for microscopic analysis. However, urine samples could include multiple cell types including red blood cells, white blood cells, eosinophils, bacteria, yeast, parasites in addition to the few tumor cells (for e.g., UTC from the kidney) from the patients. This makes it hard to detect and accurately quantitate the cancer tumor cells in urine. It can therefore be technically challenging to isolate DNA from tumor cells (for e.g., kidney cancer cells) in urine using currently available methods.

A low quantity of tumor cells and DNA exist in urine during early stages of renal cancer, so enrichment of tumor specific DNA or tumor cells is needed for accurate detection. In cancer patients, urine may contain free (noncellular, or cell-free) tumor DNA; however, there is no viable method available to separate tumor DNA from non-tumor DNA present in urine without a prior knowing the exact molecular changes in those DNA strands, and without obtaining a high ratio of DNA from diseased cells. It is a technical dilemma to separate free UTC in urine away from blood and other cells and any free DNA (for e.g., from human or microbial origin) present in urine. Additionally, a larger volume of urine is needed to capture and concentrate the tumor cells (20 to 100 ml) mostly present in low amounts.

The present disclosure is based in part on the surprising discovery that tumor cells in urine (Urinary Tumor Cells; UTC) samples from patients suffering from kidney cancer can be isolated and/or accurately quantified using a membrane filtration device provided herein. The device is non-invasive, does not involve expensive imaging such as CT-scans or patient radiation exposure, is easy to use and requires equipment present in any clinical laboratory. It can therefore be implemented more frequently and can be used for both screening and monitoring. Additionally, the method has high sensitivity that allows early detection using routine sample collection. The device disclosed herein can isolate and/or concentrate UTC in urine using a microfiltration membrane assembly to separate blood cells and other circulating or tissue free cells and free DNA in the urine from UTC. Additionally, estimate of Tumor cell or Tumor cell density (eUTC), is used herein as a potential indicator for renal cancer disease detection and progression. The disclosure further provides methods for isolating DNA from UTCs to detect novel biomarkers with mutations (for e.g., discerning sequences) or without mutations (for e.g., common human or housekeeping gene sequences) using qPCR and other techniques. The device disclosed herein is further designed with a clog-free filter which can also be used as a part of DNA isolation process by filtering or removing blood cells and cell free DNA (cfDNA) from urine, collecting and then by lysing captured tumor cells on the filter to release Urinary tumor cell DNA (utcDNA) into the filtrate. The utcDNA is purified and then used for PCR analysis for quantifying the UTC.

The detection methods provided herein can improve clinical outcomes significantly by detecting kidney cancer prior to later stage symptoms or incidental tumor discovery, by combining DNA isolation, microfiltration, and cell-specific PCR. It allows detection of kidney cancer during the early stage that is often present without symptoms. The method holds promise to improve health outcomes by increasing kidney cancer survival rate from 15% (when discovered at the late stage) to 93% (discovered at the early stage). It can be used for noninvasive preventive testing for early detection of kidney cancer in high-risk groups and can be used in conjunction with all clinical information from validated diagnostic tests, physical examination, and completed medical history. The method can also be used as a preventive test in clinics across the country and improve surveillance of kidney-based urinary tract cancers in this very high-risk group of patients (for example, the ˜55,758 people over the age of 65 in the US).

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein “Kidney cancer” refers to a disease in which cancer develops in the kidney.

As used herein the term “UTC”, or “UTCs” or “urinary tumor cells; “tumor cells”, “kidney tumor cells”, “kidney cancer cells”, “renal cancer cells” are used interchangeably to encompass cells that are shed into urine from the vasculature or lymphatics from a kidney tumor or directly into urine from a kidney tumor, irrespective to positioning of the tumor within the kidney, surrounding tissues, organs, or the urinary tract. In some aspects, the tumor cells comprise kidney cells known to be associated with kidney cancer.

As used herein the term “estimated number of tumor cells in urine” or “eUTC” refers to an estimation of number of tumor cells in urine. In some aspects, the eUTC can be tumor cell density in the urine. Tumor cell density as used herein refers to number of tumor cells in a volume of urine (for e.g., 1 ml or 50 ml).

As used herein, the term “patient”, “subject”, “subject in need thereof” or “test subject” refers to any organism from which a urine sample is sourced for use with the device provided herein. Typical subjects include animals (e.g., mammals such as mice, rats, rabbits, dogs, cats, horses, cattle, non-human primates, humans etc.). In an aspect, a subject is a human. In one aspect the subject is a human being screened for kidney cancer. In some aspects, a subject may be suffering from, and/or susceptible, predicted to or diagnosed with kidney cancer. In some aspects, the human may be of any age or gender.

As used herein, the term “target analyte” or “analyte” refers to a substance in a sample capable of being detected and analyzed by the present disclosure. Target analytes can include, but are not limited to, molecules, peptides, proteins and fragments thereof, nucleic acids, oligonucleotides, cells and fragments and products thereof, enzyme substrates, enzymes, ligands, carbohydrates, hormones, sugar, metabolic byproducts, cofactors, small molecules, biomarkers indicative of kidney cancer.

As used herein, the term “discerning DNA sequence” refers to any DNA sequence that can be used as a marker or analyte for the cancer cells (for e.g., kidney cancer cells, bladder cancer cells, urinary tract cancer cells, prostate cancer cells, or testicle cancer cells). In some aspects, it can be a mutant gene sequence, with the mutation unique to or predominant in the cancer cells. In some aspects, it can be a nucleic acid sequence from a usually dormant gene that is expressed in the cancer cells. In some aspects, it can be a nucleic acid sequence of an isoform expressed predominantly or uniquely in the cancer cells. In the latter cases the “discerning” sequence is an RNA sequence reverse transcribed to a cDNA. In some aspects, non-limiting examples of discerning sequence for use as an analyte or marker for kidney cancer may be one or more of BAP-1, PBRM1, SETD2, PIK3CA, MET gene, TP53, VHL gene, FH gene, NF2, YWHAH, CAPNS1, KNG1, SERPING1, F2, and/or PTMA. In some aspects, non-limiting examples of discerning sequence for use as an analyte or marker for bladder cancer or urinary tract cancer may be one or more of RB1, HRAS, BRCA1, BRCA2, MSH2, CHEK2, ERCC3, TSC1, FGFR3, PIK3CA, KDM6A, TP53, Coronin-1A, Apolipoprotein A-IV, Semenogelin-2, Gamma-synuclein and DJ-1. In some aspects, non-limiting examples of discerning sequence for use as an analyte or marker for prostate cancer may be one or more of BRCA1, BRCA2, HOXB13, ATM, CHEK2, MLH1, MSH2, MSH6, PMS2, PALB2, FANCA, EPCAM, HOXB13, NBN, TP53, RAD51D, MUTYH, DLX1, NKX2-3, CRISP3, PHGR1, THBS4, AMACR, GAP43, FFAR2, GCNT1, SIM2, STX19, KLB, APOF, LOC283177, and TRPM4. In some aspects, non-limiting examples of discerning sequence for use as an analyte or marker for testicle cancer may be one or more of STK11, LHR gene, TEX14, SEPT4, RAD51C, TRIM37, KRAS, NRAS, PIK3CD, PIK3CA, and CHEK2. In some aspects, the analyte, is a sequence associated with a housekeeping gene.

As used herein “Housekeeping gene” are control genes and have stable and constant expression in a wide variety of tissues or cells. In gene expression analysis, housekeeping genes are used as internal controls for standardization or normalization purposes. Many housekeeping genes are vital to the metabolism of viable cells and are therefore constantly expressed. Many housekeeping genes encode enzymes or structural RNAs, such as ribosomal RNAs, which perform essential metabolic functions and are therefore constantly expressed.

As used herein “GAPDH” or “Glyceraldehyde 3-phosphate dehydrogenase” or “GAPD” or “G3PD” or “HEL-S-162eP” refers to an enzyme of about 37 kDa that catalyzes the sixth step of glycolysis and thus serves to break down glucose for energy and carbon molecules. Based on these basic and ubiquitous cellular functions, GAPDH is considered as a housekeeping gene. GAPDH is encoded by a single gene that produces a single mRNA transcript with 8 splice variants, though an isoform does exist as a separate gene that is expressed only in spermatozoa. Nucleotide sequences of GAPDH are available on the world wide web from the NCBI, including human, mouse, and rat. For example, the nucleotide sequence encoding human GAPDH gene is available at NCBI under Accession No. NG_007073.2, and nucleotide sequence of a transcript variant (for e.g., Homo sapiens glyceraldehyde-3-phosphate dehydrogenase (GAPDH), transcript variant 1, mRNA) is available at NCBI under Accession No. NM_002046.7. Primers and probes for detection of GAPDH can be designed and optimized using methods known in the art. Methods of detection of a gene or mRNA sequences of GAPDH present in a sample is well known in the art, for e.g., PCR, qPCR, RT-PCR, etc.

As used herein “Aggressiveness” of kidney cancer or a cancer-positive small renal mass refers to a combination of the stage, grade, and metastatic potential of a kidney tumor. “More aggressive” kidney cancer refers to tumors of higher stage, grade, and/or metastatic potential. Cancer tumors that are not confined to the kidney are considered to be more aggressive kidney cancer. “Less aggressive” kidney cancer refers to tumors of lower stage, grade, and/or metastatic potential.

As used herein “Staging” of kidney cancer refers to an indication of the severity of kidney cancer including tumor size and whether and/or how far the kidney tumor has spread. The tumor stage is a criteria used to select treatment options and to estimate a subject's prognosis. Kidney tumor stages range from T1 (tumor 7 cm or less in size and limited to kidney, least advanced) to T4 (tumor invades beyond Gerota's fascia or progressed to distant sites as metastatic disease, most advanced). “Low stage” or “lower stage” as well as the grade (grades I-IV) classification of cells within the kidney cancer tumor refers to kidney cancer tumors, including malignant tumors with a lower potential for recurrence, progression, invasion and/or metastasis (less advanced). Kidney tumors of stage T1 or T2 are considered “low stage”. “High stage” or “higher stage” kidney cancer refers to a kidney cancer tumor in a subject that is more likely to recur and/or progress and/or invade beyond the kidney, including malignant tumors with higher potential for metastasis (more advanced). Kidney tumors of stage T3 or T4 are considered “high stage”. The WHO/ISUP grade of tumor cells also plays a significant role in tumor metastatic potential, as this signifies aggressiveness and invasiveness of the cells within the tumor. Tumors with cells of grades 1-2 are slower growing and less invasive into surrounding tissues than tumors with cellular grades 3-4; therefore, identifying presence of tumors in the early stage can also catch aggressive high-grade tumors while they are small, before they damage or invade surrounding tissues within the kidney. Early detection reduces the chance of cancer recurrence or metastasis to distal parts of the body. Kidney cancer, particularly clear cell Kidney Cancer can metastasize to any part of the body, and can recur many years to decades after the initial treatment, requiring routine screening and monitoring after initial diagnosis.

In some aspects, the Staging System of the American Joint Committee on Cancer (AJCC) known as the TNM system may be used. The letter T followed by a number from 1 to 3 describes the tumor's size and spread to nearby tissues. Higher T numbers indicate a larger tumor and/or more extensive spread to tissues near the kidney. The letter N followed by a number from 0 to 2 indicates whether the cancer has spread to lymph nodes near the kidney and, if so, how many are affected. The letter M followed by a 0 or 1 indicates whether or not the cancer has spread to distant organs.
Stage I: The tumor is 7 cm (about 2¾ inches) or smaller, and limited to the kidney. There is no spread to lymph nodes or distant organs.
Stage II: The tumor is larger than 7.0 cm but still limited to the kidney. There is no spread to lymph nodes or distant organs.
Stage III: Includes tumors of any size, with or without spread to fatty tissue around the kidney, with or without spread into the large veins leading from the kidney to the heart, with spread to one nearby lymph node, but without spread to distant lymph nodes or other organs. Stage III also includes tumors with spread to fatty tissue around the kidney and/or spread into the large veins leading from the kidney to the heart, that have not spread to any lymph nodes or other organs.
Stage IV: This stage includes any cancers that have spread directly through the fatty tissue and the fascia ligament-like tissue that surrounds the kidney. Stage IV also includes any cancer that has spread to more than one lymph node near the kidney, to any lymph node not near the kidney, or to any other organs such as the lungs, bone, or brain.

As used herein “amount” or “level” refers to the abundance of a particular analyte (e.g., GAPDH, or tumor cells) present in the sample. A used herein “expression” or “expression level” or “level of expression” refers to the level of a particular analyte in a urine sample or filter retentate, for example: DNA or RNA. The amount may be a number, ratio, proportion, or a percentage of the analyte compared to the control sample or determined using a standard curve. The amount may be an absolute amount or a relative amount.

As used herein, treatment of cancer can comprise administration of a therapy for the purposes of increased inhibition of cancer progression and/or metastases, inhibition of an increase in tumor volume, a reduction in tumor volume and/or growth, a reduction in tumor growth rate, an eradication of a tumor and/or cancer cell, reduction in the chance or incidence of tumor recurrence, or any combination thereof. In some aspects, the treatment can also prolong the survival of a subject, improve the prognosis and/or improve the quality of life of the subject.

In some aspects, the treatment of kidney cancer as used herein can comprise administration of compounds, including for example, chemotherapeutic agents, anti-inflammatory agents, anti-pyretic agents radiosensitizing agents, radioprotective agents, urologic agents, anti-emetic agents, and/or anti-diarrheal agents, a platinum based anti-neoplastic agent, a topoisomerase inhibitor, a nucleoside metabolic inhibitor, an alkylating agent, an intercalating agent, a tubulin binding agent, for example, cisplatin, carboplatin, docetaxel, paclitaxel, fluorouracil, capecitabine, gemcitabine, irinotecan, topotecan, etoposide, mitomycin, gefitinib, vincristine, vinblastine, doxorubicin, cyclophosphamide, celecoxib, rofecoxib, valdecoxib, ibuprofen, naproxen, ketoprofen, dexamethasone, prednisone, prednisolone, hydrocortisone, acetaminophen, misonidazole, amifostine, tamsulosin, phenazopyridine, ondansetron, granisetron, alosetron, palonosetron, promethazine, prochlorperazine, trimethobenzamide, aprepitant, diphenoxylate with atropine, and/or loperamide. In some aspects, treatment can comprise administration of antiangiogenetic drugs, including for example monoclonal antibodies directed against Vascular Endothelial Growth Factor (VEGF) and Placental Growth Factor (PIGF); and inhibitors of the VEGF and PIGF receptors, including for example bevacizumab, sorafenib, PTK78, SU11248, AG13736, AEE788, and ZD6474. In another aspects, the treatment comprises administration with immunomodulatory drugs, including for example interleukin 2 (IL-2) and Interferon alpha (IFNα), PD-1 inhibitors, checkpoint inhibitor immunotherapy, immunotherapy drugs such as ipilimumab, nivolumab, sunitinib, cabozantinib, and pembrolizumab, and combinations therein, for examples. In another aspect, treatment comprises administering drugs interfering with cellular growth signaling, including for example inhibitors of the mammalian target of rapamycin (mTOR). In some aspects, the treatment may involve radiation treatments. In some aspects, the treatment comprises surgical removal of the tumor, tumor ablation, energetic disruption, cryo-treatment, heat treatment, or removal of the afflicted kidney (radical nephrectomy) or a portion thereof (partial nephrectomy).

As used herein a “reference” or a “control sample” is a sample which may be procured from a healthy subject. In some aspects, the reference can comprise of an average levels of the target analyte, amount of UTC or tumor cell density in a sample from a subject before onset of cancer. In some aspects, a reference sample can be a sample from the subject prior to diagnosis or treatment. In certain aspects, the levels of the target analyte, amount of UTC or tumor cell density can be measured in a person or persons other than the subject with cancer. In some aspects, the reference is a person or persons with similar characteristics to the subject with cancer. In some aspects, the reference can be an average of the combination of disclosed levels of the target analyte, amount of UTC or tumor cell density from different healthy sources (e.g., more than one healthy control subject). In some aspects, the control sample can be pooled sample.

### I. Device

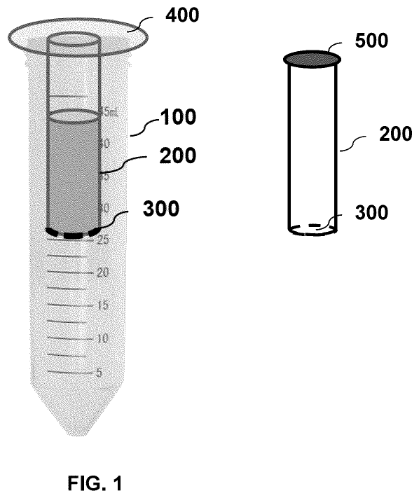

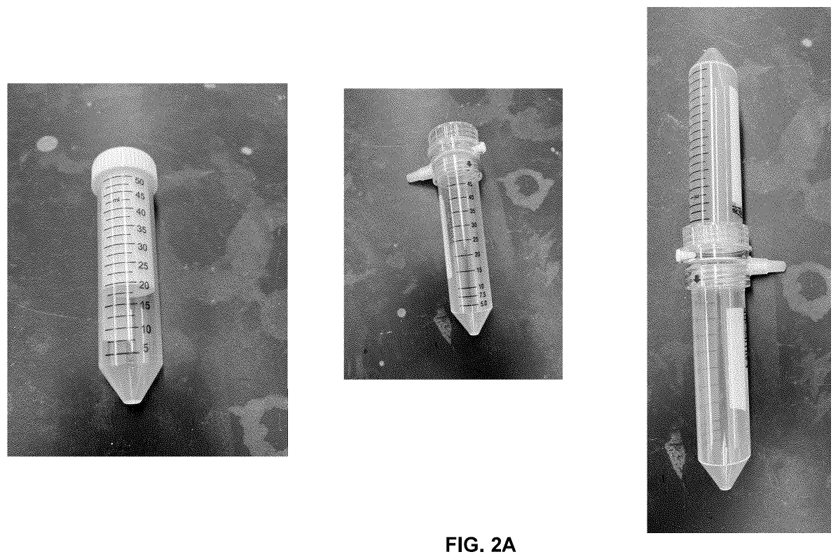

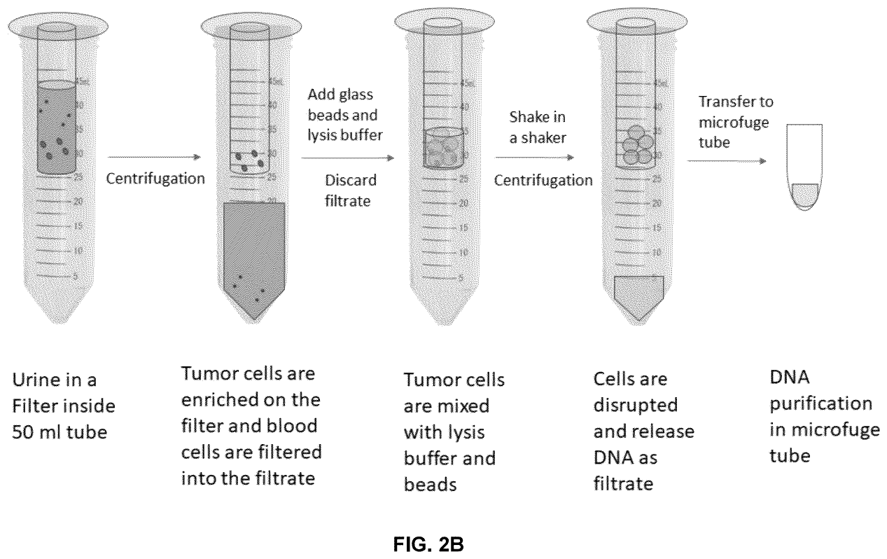

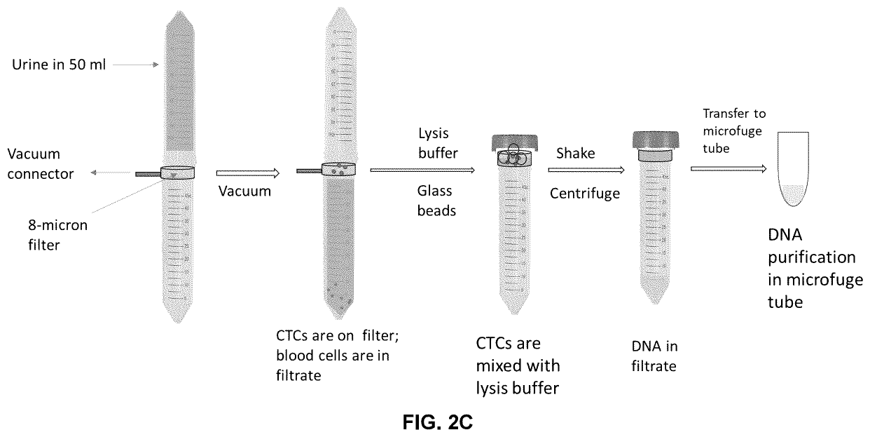

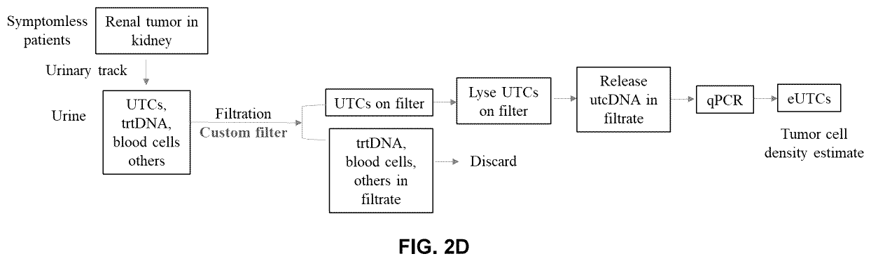

An aspect of the present disclosure encompasses a microfiltration device to isolate DNA from Urinary Tumor Cells (UTCs) in urine for kidney cancer. The device described herein is configured to further isolate a target analyte (for e.g., DNA or RNA). Various configurations of the assembled device are shown in FIG. 1 and FIG. 2A. In one exemplary aspect, the use of the device is shown in FIG. 2B-2D.

In some aspects, the device comprises a container member (100) also referred to as the collection tube which is shown as a 50 ml conical tube in this exemplary aspect. However, the current disclosure encompasses container member of any suitable shape including but not restricted to flat, rounded, or curved edged. In some aspects, the container member is adapted to be used with a centrifuge or a vacuum manifold or pump. In some aspects, the device can comprise a connector adapted for use with a vacuum device or a vacuum pump. In some aspects, the container member may be adapted to be used with any suitable centrifuge including but not restricted to a microcentrifuge, a bench top centrifuge, a clinical centrifuge, a floor centrifuge, or an ultracentrifuge. In some exemplary aspects, the container member has dimensions such that it can fit into a holder of one or more of commercially available centrifuges. In some exemplary aspects the container member has the dimension of a microcentrifuge tube.

In some exemplary aspects, the container member has the overall dimensions equivalent to any commercially available tubes (for example 1.5 mL, 15 mL or 50 mL) that fit into a holder of a bench top centrifuge. In some exemplary aspects, the container member has the dimensions equivalent to any commercially available tube that fit into a holder for a clinical centrifuge. In some exemplary aspects, the container member fits into one or more of commercially available vacuum manifolds including but not limited to Vac-Man® (Fischer Scientific), Aurum™, HyperSep™ (Thermo Scientific), EZ-Vac™ (Thomas Scientific), Millipore Sigma Steriflip. In some aspects, urine may be obtained using any commercially available urine collection container or cup. In such aspects, a required amount of urine sample may be transferred to the container member of the disclosed device for further analysis.

In some aspects, the collection tube may comprise any suitable material for example a glass, polypropylene, polyethylene, polycarbonate, polymer, metal, any suitable plastic, or combinations thereof. In some aspects, the collection tube may comprise a lid (400). In some aspects, the lid may be attached to the container member. In some aspects the lid may be detachable from the container member.

In some aspects, the device comprises a first filtration member (200) concentrically positioned inside the container member. In some aspects, the first filtration membrane may be of any suitable shape. In some aspect, as provided in the FIG. 1, the first filtration member is of a cylindrical shape as provided in the figure. In some aspects, the first filtration member is conical in shape similar to the container member. In some aspects, the first filtration member is cylindrical in shape with an essentially flat, rounded, or conical bottom. In some aspects, the first filtration member may comprise any suitable material for example glass, polypropylene, polyethylene, polycarbonate, polymer, metal, any suitable plastic, polyethersulfone, polysulfone, polyethylene, UHMWPE, PET (Polyethylenterephthalat), polyester (PETE), polycarbonate track etch, cellulose, mixed cellulose esters, cellulose acetate, cellulose nitrate, hydrophilized polyvinylidene fluoride, gel, silica, alumina, or any combination thereof. In some aspects, the first filtration member is made of ultra-high molecular weight polyethylene with the advantage that it Scan be used with glass beads.

In some aspects, the filtration member may comprise a porous membrane, a filter, a screen, any material is fibrous or porous and are able to capture UTC. In some aspects, the fibrous or porous material may be a cellulosic material, a filter paper material, absorbent textured material, absorbent sintered materials, absorbent pastes, microporous membranes, sponge, film, fibers, matrix, fiberglass, and the like.

In some aspects, the filtration member is a charged commercially available membrane. In some aspects, the filtration member is neutral. In some aspects, the filtration member is selected from Mustang® Ion Exchange Membrane from PALL Corporation; Vivapure® Q membrane from Sartorius AG; Sartobind Q, or Vivapure® Q Maxi H; Sartobind® D from Sartorius AG, Sartobind(S) from Sartorius AG, Sartobind® Q from Sartorius AG, Sartobind® IDA from Sartorius AG, Sartobind® Aldehyde from Sartorius AG, Whatman® DE81 from Sigma, Fast Trap Virus Purification column from EMD Millipore; Thermo Scientific® Pierce Strong Cation and Anion Exchange Spin Columns. In some aspects, filtration member is a Q PES vacuum filtration (Millipore), 3-5 μm positively charged Q RC spin column filtration (Sartorius), 0.8 μm positively charged Q PES homemade spin column filtration (Pall), 0.8 μm positively charged Q PES syringe filtration (Pall), 0.8 μm negatively charged S PES homemade spin column filtration (Pall), 0.8 μm negatively charged S PES syringe filtration (Pall), or 50 nm negatively charged nylon syringe filtration (Sterlitech). In further aspects, the filter membrane may be functionalized (for e.g., quaternary ammonium, sulfonic acid acid groups, diethylamine groups, etc.).

In some aspects, the filtration member comprises a first filtration membrane (200) and (300) with a pore size of about 5 to 20 microns. In some aspects, the membrane has a pore size of about 5, 6, 7, 8, 9, 10, 11, 12, 13, 14, or 15 microns. In some aspects the membrane pore size is such that it can retain tumor cells for example kidney tumor cells, or UTC, while allowing smaller cells and particulates like blood cells, bacterial cells and viruses to pass through. In some aspects, a filtration membrane with a smaller pore size about 0.1 to 1 micron may be used in the disclosed device. In some aspects, the membrane has a pore size of about 0.1, 0.2, 0.3, 0.4, 0.5, 0.6, 0.7, 0.8, 0.9, or 1 micron. For capturing UTC, a pore size of about 8 microns to 20 microns may be used. In some aspects, the pore-size may range from about 8 microns to about 12 microns. In some aspects, the pore size of the first filtration membrane is about 8 microns.

In some aspects, as provided in FIG. 1 the filtration membrane may line the bottom of the first filtration member. In some aspects, the filtration membrane may line the sides of the first filtration member. In some aspects, the filtration membrane may line a partial surface of the first filtration member. In some aspect, the filtration membrane may line the entire surface of the first filtration member.

In some aspects of the device, the filtration membrane comprise a polymer selected from a group consisting of polyethersulfone, polysulfone, polypropylene, polyethylene, UHMWPE, PET (Polyethylenterephthalat), polyester (PETE), polycarbonate, polycarbonate track etch, cellulose, mixed cellulose esters, cellulose acetate, cellulose nitrate, hydrophilized polyvinylidene fluoride, or any combination thereof.

In some aspects, the filtration member is removable from the container member. In some aspects, the filtration member can be placed in a second container member after removal.

In some aspects, the filtration member has a cap or a washer that can fit inside the cap of 50 ml tube. In some aspects, the cap may be detachable or attached to the filtration member. In some aspects, the cap may be made of any suitable material including glass, polypropylene, polyethylene, polycarbonate, polymer, metal, any suitable plastic, or any combination thereof.

In some aspects, the device may further comprise a strainer of pore size larger than the first filtration membrane. In some aspects, the strainer is concentrically positioned inside the first filtration member to prevent clogging. In some aspects, the strainer may be removable. In some aspects, the strainer may be made of any suitable material for example glass, polypropylene, polyethylene, polycarbonate, polymer, metal, any suitable plastic, polyethersulfone, polysulfone, polycarbonate track etch, cellulose, mixed cellulose esters, cellulose acetate, cellulose nitrate, hydrophilized polyvinylidene fluoride.

In some aspects, the device may comprise a second filtration member concentrically positioned outside or inside the first filtration member as is suitable for the desired application. In some aspects, the second filtration member is positioned inside the first filtration member and comprises a second filtration membrane of a pore size greater than the first filtration membrane. In some aspects, the second filtration member may have a pore size smaller than the first filtration membrane and may retain particulates or other components of the sample which have passed through the first filtration membrane. In some aspects, the device may comprise a plurality of filtration membrane filters which are disposed sequentially in the container membrane. In such aspects, each of the membrane filters may have a pore size sequentially greater or smaller.

In some aspects, the device may comprise a second container member or a collection tube, as depicted in FIG. 2A and FIG. 2C. In such aspects, the second container membrane or collection tube is positioned atop the first container member, and can comprise a membrane filtration member positioned between the first container member and second container member. In further aspects, the device can further comprise a connector adapted for use with a vacuum device or a vacuum pump positioned between the first container and second container for use in moving the sample through the filter from the second container member to the first container membrane.

In further aspects, the device disclosed herein may further comprise a plurality of ports and the ports may be in fluid communication with various members of the device. In such aspects, the ports may be used for introduction of reagents, and for the removal of waste products and/or extracted nucleic acid. For example, a first port can be used for introducing a urine sample into the first container member and a second port may be used for removing the urine sample after isolation of UTC.

In further aspects, the device may further comprise reagents or components for use in lysing UTC (for e.g., beads), isolating target analyte (DNA or RNA), detection and quantification of target analyte (for e.g., reagents for qPCR).

### II. Method

In some aspects, the current disclosure encompasses methods for isolating detecting, and/or determining UTC in urine samples linked to kidney cancer.

In some aspects, the urine sample from the subject can be procured one or more times, before, during and/or after diagnosis of kidney cancer. In some aspects, urine samples can be procured from the subject before, during, and/or after treatment of kidney cancer. In some aspects, urine sample can be procured from the subject prior to the start of the cancer treatment. In some aspects, sample can be procured from the subject undergoing cancer treatment. Additionally, urine samples can be procured repeatedly at multiple stages after initial sample procurement, to determine and/or monitor kidney cancer in a subject.

In some aspects of the current disclosure, the subject is a mammal who is being screened for, suspected of, predicted to or diagnosed with kidney cancer. In an aspect, a subject is a human. In one aspect the subject is a human being screened for kidney cancer. In some aspects, a subject may be suffering from, and/or susceptible, predicted to or diagnosed with kidney cancer. In some aspects, the human may be of any age or gender. Urine samples can be obtained from the subject and stored by any method known in the art. In some aspect, the sample may be used directly without storage. In some aspects the urine sample may be appropriately stored. In some aspects, the urine sample may be pre-processed, for example concentrated, strained, filtered or centrifuged prior to application to the device disclosed herein. In some aspects, the urine sample may be subjected to a concentration step prior to application to the device. In some aspects, the urine sample may be subjected to a centrifugation step prior to application to the device. In some aspects, where the goal of the centrifugation step was to remove particulate matter, the supernatant may be applied to the device. In some aspects, wherein the goal is to concentrate cells, the pellet may be resuspended in a suitable buffer and applied to the device to separate tumor cells from other cells. In some aspects, additives like antimicrobials, preservatives, or buffering agents may be added to the sample.

In some aspects, the urine sample that is obtained from a subject is applied to the device provided herein. In some aspects, the urine sample may be a fresh urine sample, a processed urine sample, or a stored urine sample. The amount of urine sample that is passed through the device can vary, for example depending on the size of the device, scale of analysis, and the detection method used. In some aspects, the amount of urine needed may vary from microliter scale to about 500 mL. In some aspects, the volume may be vary between about 50 μL to about 5 mL. In some aspects, the volume may vary between 5 mL to about 15 mL, or about 15 mL to about 50 mL, or about 50 mL to about 100 mL, or about 10 mL to about 500 mL. In some aspects, about 50 ml of urine sample is used in the disclosed methods. In some aspects, an initial larger volume of urine may be obtained, and may then be subdivided for analysis. In some aspects, and depending on the scale of the device, multiple application of sample may be needed and is envisaged in the method.

In some aspects, once the sample is applied to the device, a positive pressure may be needed to pass the sample through the strainer, and/or a first filtration member, and/or a second filtration member. In some aspects, the positive pressure may be applied using a centrifugation step. The centrifuge and speed of centrifugation can vary depending on the size and materials (for example the material used to make the filtration membrane) incorporated in the device. Non-limiting examples of appropriate centrifuge that can be used include a microcentrifuge, a bench top centrifuge, a clinical centrifuge, a floor centrifuge, or an ultracentrifuge. In some aspects, the positive pressure may be applied using a vacuum. In some aspects, the positive pressure may be applied using a vacuum manifold linked to a vacuum pump. Non limiting examples of vacuum manifolds that can include Vac-Man® (Fischer Scientific), Aurum™, HyperSep™ (Thermo Scientific), EZ-Vac™ (Thomas Scientific).

In some aspects of the disclosed method, on passing through the first filtration member, the UTC present in the urine sample are retained on the first filtration membrane while other cells and cell debris including for example blood cells, microbial cells, nucleic acids, proteins, lipids pass through into the supernatant.

In some aspects, the tumor cells UTC comprise kidney cells known to be associated with kidney cancer. In some aspects, the current disclosure stems from the surprising result that tumor cells associated with kidney cancer may be isolated from urinary samples in early cancer patients in detectible amounts using the device disclosed herein.

In some aspects, tumor cell density (eUTC) of the tumor cells isolated using the disclosed methods and device can be used as a biomarker for diagnosis of kidney cancer. In such aspects, the isolated UTC are characterized by the number isolated or concentration in the sample. In some aspects, the target analyte copy/ml obtained from analysis of the target analyte from the isolated UTC can be used to estimate the UTC number or density per ml in the urine.

In some aspects, the UTC are characterized by their phenotype to determine the likelihood of metastasis or the presences of a malignant tumor. Methods of characterizing the phenotype of cells with respect to their potential for metastasis are well known in the art.

In some aspects, of the current disclosure, the UTC retained on the filtration member can be resuspended in a suitable buffer system and removed from the membrane for further analysis. In some aspects, the further analysis may comprise one or more of microscopic analysis, lysis, analyte analysis, FACS analysis and/or quantification of UTC or derived analytes. In some aspects, the UTC may be retained on the filter and lysed in situ for further analysis.

In some aspects, the filtration member may be removed and put in a second container member or a collection tube. UTC retained on the filter may be lysed using known methods in the art. In some aspects, lysing the cells comprises adding a lysis solution to the retentate suspected of comprising UTC. Any of the lysis solutions known in the art may be used for this purpose. In some aspects, the lysis solution comprises one or more of buffers, detergents, salts, or DNAse inhibitors, protease inhibitor, RNAase inhibitor. In various aspects, lysing the cells comprises agitating the UTC in the retentate presence of beads, granules, pellets or other solid material suitable for disrupting cell membranes. Any bead available to facilitate the lysis of cells can be used. For example, lysing the cells can comprise using glass beads, chrome beads, steel beads, chrome-steel beads, silicon carbide beads, garnet beads, stainless steel beads, granules, pellets, plastic beads, ceramic beads, or a combination of any thereof. The agitation can comprise mechanical agitation methods including, but not limited to, sonication, magnetic, forced gas, etc. In various embodiments, the beads are glass beads. The beads can be of any size conventionally used in the art. For example, the beads can be from about 0.5 to 5 mm. In various aspects, the filter used to obtain the processed test sample does not bind nucleic acids or poorly binds nucleic acids such that a filtrate formed from filtering the original test sample through the filter comprises a majority of free nucleic acid present in the original test sample.

In some aspects, the lysed solution may be used directly for estimating a target analytes. In some aspects, the lysed solution can be used to obtain, for example DNA, RNA, protein, lipids and other molecules from the sample. These can then be used for further analyses and estimation of target analytes. In some aspects, the current disclosure encompasses quantitation of UTC using available methods for example FACS or microscopic analysis. In additions to other target analytes derived from the UTC may be used to provide diagnosis and staging information. Target analytes can include, but are not limited to, molecules, peptides, proteins and fragments thereof, nucleic acids, oligonucleotides, cells and fragments and products thereof, enzyme substrates, ligands, carbohydrates, hormones, sugar, metabolic byproducts, cofactors, small molecules, biomarkers indicative of kidney cancer. In some aspects, the fluid sample is a urine sample. In some aspects, the target analyte may be a house keeping gene used to quantify the number of cells in the urine. In some aspects, the target analyte is a wildtype gene or fragments thereof known to have distinctive expression pattern in tumor cells compared to healthy cells. In some aspects, the target analyte is a mutant gene or fragment thereof, with known pattern of mutations associated with tumor cells. In some aspects, the target analyte is a DNA sequence. In some aspects the target analyte is an RNA sequence. In some aspects, the target analyte may be a protein sequence.

In some aspects, the target analyte is a nucleotide (e.g., DNA or RNA) sequence of a housekeeping gene. Non-limiting examples of target analyte include Glyceraldehyde-3-Phosphate Dehydrogenase (GAPDH), β-actin (ACTB), 28S RNA, 18S RNA, ribonuclear protein genes, Peroxiredoxin 6 (PRDX6), ribosomal protein L37a (RPL37A), Adducin 1 (ADD1), human leukocyte antigen (HLA-A), Cell cycle checkpoint control protein RAD9A (RAD9A), Rho Guanine Nucleotide Exchange Factor 7 (ARHGEF7), eukaryotic initiation factor-2B2 (EIF2B2), 26S proteasome non-ATPase regulatory subunit 7 (PSMD7), Branched Chain Amino Acid Transaminase 2 (BCAT2), ATP synthase subunit O, mitochondrial (ATP50), Porphobilinogen deaminase (PBGD), beta-2-microglobulin (B2M), Tubulin beta chain (TUBB), Ribosomal Protein Lateral stalk subunit PO (RPLPO), Succinate Dehydrogenase Complex Flavoprotein Subunit A (SDHA), peptidyl-prolyl cis-trans isomerase (PPIA), TATA-Box Binding Protein (TBP), Hydroxymethylbilane synthase (HMBS), 5′-aminolevulinate synthase 1 (ALAS1), hypoxanthine phosphoribosyltransferase 1 (HPRT1), ribosomal RNA, alpha tubulin, enolase (for e.g., enolase 1), beta globin, karyopherin alpha 6, membrane cofactor protein, phosphoglycerate kinase, ribosomal protein L7, ribosomal protein S9, ribosomal protein S14, hypoxanthine phosphoribosyl transferase 1, ribosomal protein large PO, peptidylpropyl isomerase A (cyclosporin A), cytochrome C, phosphoglycerate kinase 1, β-glucuronidase, TATA box binding factor, transferrin receptor, HLA-A0201 heavy chain, ribosomal protein L19, a tubulin, β tubulin, y tubulin, ATP synthetase, eukaryotic translation elongation factor 1 gamma (EEF1G), succinate dehydrogenase complex (SDHA), ADP-ribosylation factor 6, endonuclease G (ENDOG), peroxisomal biogenesis factor (PEX), hexose-6-phosphate dehydrogenase (H6PD), kinesin family member IB (KIF1B), Nicotinamide nucleotide adenylyl transferase 1 (NMNAT1), ubiquitination factor E4B (UBE4B), aconitase 1 (ACO1), kelch-like 9 (KLHL9), pantothenate kinase 1, kinase family member 20B (KIF20B), or any combination thereof. In some aspects, the housekeeping gene may comprise one or more of the genes disclosed in U.S. Patent Publication No. US20040229233 A1, journal article Hounkpe et al. (Nucleic Acids Res. 2021 Jan. 8; 49 (D1): D947-D955), the disclosures of which are incorporated by reference in its entirety.

In some aspects, the target analyte is a nucleotide (e.g., DNA or RNA) sequence of a kidney specific or kidney tumor gene or target sequence. In such aspects, the target analyte is a BAP-1, PBRM1, SETD2, PIK3CA, MET gene, TP53, VHL gene, FH gene, NF2, YWHAH, CAPNS1, KNG1, SERPING1, F2, PTMA, or any combination thereof. In some aspects, the target analyte is a nucleotide (e.g., DNA or RNA) sequence of a housekeeping gene. Nonlimiting examples of target analyte include GAPDH (glyceraldehyde-3-phosphate dehydrogenase), aquaporin 1 (AQP1), adipophilin (ADFP), NADPH oxidase 4 (Nox 4). In some aspects, the target analyte is a nucleotide (e.g., DNA or RNA) of GAPDH.

In some aspects, quantitation of target analyte can be achieved by any known method in the art. In some aspects, the methods provided herein can comprise subjecting the isolated DNA/RNA to a polymerase chain reaction (PCR) to amplify the nucleic acid corresponding to a target analyte from the lysed cells. Any polymerase chain reaction known in the art can be used to amplify the nucleic acids. In various aspects, the polymerase chain reaction comprises Real-time PCR (quantitative PCR or qPCR), Reverse-Transcription PCR (RT-PCR), Multiplex PCR, Nested PCR, High Fidelity PCR, Fast PCR, Hot Start PCR, Long-range PCR, Arbitrary Primed PCR, Digital PCR, Droplet Digital PCR (ddPCR), isothermal amplification PCR, Endpoint PCR (qualitative PCR), or a combination of any thereof. In various aspects, the polymerase chain reaction comprises of qPCR or ddPCR.

Each of the PCR reactions described above can be performed according to standard methods in the art, including those provided in laboratory manuals such as Sambrook, J., et al, Molecular Cloning: A Laboratory Manual, 3rd ed., Cold Spring Harbor Laboratory Press, Cold Spring Harbor, N.Y., 2001; Spector, D. L., et al., Cells: A Laboratory Manual, Cold Springs Harbor Laboratory Press, Cold Spring Harbor, N.Y., 1998; Carruthers, W., and Coldham, I., Modern Methods of Organic Synthesis (4th Edition), Cambridge University Press, Cambridge, UK., 2004, herein incorporated by reference in its entirety.

As noted, the methods provided herein comprise analyzing an amplified nucleic acid produced by a PCR protocol or whole or partial genome of the isolated UTC. In various aspects, the analysis can comprise sequencing the nucleic acid using, but not limited to, Sanger Sequencing, single molecular real time (SMRT) sequencing, nanopore DNA sequencing, massively parallel signature sequencing (MPSS), colony sequencing, 454 pyrosequencing, Illumina sequencing, combinatorial probe anchor synthesis (cPAS), SOLID sequencing, ion torrent semiconductor sequencing, DNA nanoball sequencing, helioscope single molecule sequencing, using a microfluidic system or a combination of any thereof.

In other aspects, the analysis can comprise quantitative detection of the nucleic acid, such as using an oligonucleotide probe or nucleic acid dye. In various aspects, the quantitative detection occurs as part of the polymerase chain reaction performed (e.g., using real time PCR or quantitative PCR (qPCR). In other aspects, the quantitative detection occurs after the polymerase chain reaction is performed. For example, in some aspects, the quantitation can use a nanopore or similar nanotechnology to detect the nucleic acids. See for example Kang et al., “Ready-to-use nanopore platform for the detection of any DNA/RNA oligo at attomole range using an Osmium tagged complementary probe” Scientific Reports 10, 19790 (2020), which is incorporated herein by reference in its entirety. In still further aspects, the analysis can comprise qualitative detection, such as using an agarose gel, polyacrylamide electrophoresis, restriction endonuclease digestion, dot blots, liquid chromatography, electrochemiluminescence, or any combination thereof. In general, any method known in the art to sequence, quantify, or detect the nucleic acid may be used in the methods provided herein. In any aspect provided herein, identifying the nucleic acid as indicative of the presence of the intact cell of interest is contemplated.

In various aspects, the current disclosure also encompasses screening for non-nucleic acid analytes including but not limited to proteins, peptides, toxins, metabolites and small molecules from the samples. In some aspects, the screening for proteins/peptides may involve one or more of a western blot, ELISA, mass spectrometry, chromatography, and FACS for surface expressed proteins. In some aspects the screening for metabolites, small molecules may include for example, chemical analysis or mass spectrometric analysis.

In some aspects, assaying, screening for or analysis of a target analyte includes both qualitatively or quantitatively measuring or estimation. In some aspects, the assaying can be direct or relative to another sample. Therefore, in some aspects, the current disclosure further comprises comparing the levels, number, and/or amount of a target analyte with levels, number and/or amount of a target analyte found in a similarly processed sample from the subject, a patient, a population of patients, or a healthy individual, a population of healthy individuals, or an established ‘expected value’ baseline based on healthy individual or population of individuals. As used herein the term “healthy individual” is used in a restricted sense to include individuals who are not diagnosed with or suspected to have kidney cancer. In some aspects, the levels, number, and/or amount of a target analyte from a population of healthy individual is averaged to be used as a threshold. Deviation from the threshold level may be used to assist in making a determination, typing or staging of kidney cancer. In some aspects, the current method can be used for monitoring disease progression or treatment outcomes in a subject in need thereof. In such cases a comparison may be made of levels/number/amount of a target analyte at a given time with previously obtained values.

In some aspects, the disclosed method use the quantity of target analyte (e.g., GAPDH), amount or density of UTC (eUTC) is used in the methods disclosed herein.

In some aspect, the methods disclosed herein comprise the steps of (a) obtaining or having obtained the urine sample; (b) passing the urine sample through a device as provided herein to obtain a retentate, wherein the retentate is enriched in UTC compared to the urine sample; (c) detecting the target analyte present in the retentate.

In some aspects, the method encompasses a method for classifying a subject suspected or at risk of having kidney cancer. In such aspects, the method comprises obtaining or having obtained a urine sample from the subject; passing the urine sample through a filtration member to obtain a retentate, wherein the retentate is enriched in UTC compared to the urine sample; lysing or disrupting the intact cells in the retentate to form a lysed/disrupted cell mixture comprising nucleic acid from lysed/disrupted cells; filtering the lysed/disrupted cell mixture through the filter to form a filtrate comprising the nucleic acid from the lysed/disrupted cells; subjecting the filtrate from (d) to a polymerase chain reaction (PCR) to amplify the nucleic acid and to quantify the level of a target analyte (for e.g., GAPDH DNA or a fragment thereof) present in the retentate; comparing the level of the target analyte in the urine sample to a reference level of the target analyte in a control sample; using that value to estimate the number of UTC (eUTC) in the sample, detecting the presence of kidney cancer in the subject if the level of the target analyte (eUTC) in the urine sample is elevated compared to the reference level; classifying the subject as a candidate for a kidney cancer therapy or in-depth clinical workup and imaging under suspicion of kidney cancer prior to therapy, based on the level of the target analyte.

In some particular aspects, the method is a method for detecting the presence of kidney cancer from a urine sample from a subject at risk or suspected of having kidney cancer. In such aspects, the method comprises obtaining or having obtained the urine sample from the subject; passing the urine sample through a filtration member comprising a membrane having a pore size of about 8 microns to obtain a retentate, wherein the retentate is enriched in UTC cells compared to the urine sample; lysing or disrupting the intact cells in the retentate to form a lysed/disrupted cell mixture comprising nucleic acid from lysed/disrupted cells; filtering the lysed/disrupted cell mixture from (c) through the filter to form a filtrate comprising nucleic acid from the lysed/disrupted cells; detecting the presence of a mammalian housekeeping gene such as GAPDH DNA or a fragment thereof in the filtrate, and using that value to estimate the number of UTC (eUTC) in the sample. In such aspects, the subject at risk or suspected of having kidney cancer may have elevated level of GAPDH DNA or a fragment thereof compared to the reference level. In some aspects, the subject at risk or suspected of having kidney cancer may have elevated level of UTC or tumor cell density (eUTC) as compared to the reference level. In such aspects, the reference level may be level of or average level of the target analyte, amount of cells or cell density in the urine sample of a subject or subjects without cancer.

In some particular aspects, the method is a method for early-stage detection of kidney cancer from a urine sample from a subject at risk or suspected of having kidney cancer. In such aspects, the method comprises obtaining or having obtained the urine sample from the subject; passing the urine sample through a filtration member comprising a membrane having a pore size of about 8 microns to obtain a retentate, wherein the retentate is enriched in UTC cells compared to the urine sample; lysing or disrupting the intact cells in the retentate to form a lysed/disrupted cell mixture comprising nucleic acid from lysed/disrupted cells; filtering the lysed/disrupted cell mixture from (c) through the filter to form a filtrate comprising nucleic acid from the lysed/disrupted cells; detecting the presence of a mammalian housekeeping gene such as GAPDH DNA or a fragment thereof in the filtrate, and using that value to estimate the number of UTC (eUTC) in the sample. In such aspects, the subject at risk or suspected of having kidney cancer may have elevated level of GAPDH DNA or a fragment thereof compared to the reference level. In some aspects, the subject at risk or suspected of having kidney cancer may have elevated level of UTC or tumor cell density (eUTC) as compared to the reference level. In such aspects, the reference level may be level of or average level of the target analyte, amount of cells or cell density in the urine sample of a subject or subjects without cancer. In some aspects, early stage cancer is a stage or grade 1 or 2 cancer.

In further aspects, the disclosure provides a method for monitoring a subject having a kidney cancer. In such aspects, the method comprises obtaining urine sample from the subject, wherein the urine samples are obtained before, during and/or after administration of a cancer treatment. The samples obtained are then passed through the filtration member comprising a membrane having a pore size of about 8 microns to obtain a retentate, wherein the retentate is enriched in UTC compared to the urine sample; lysing or disrupting the intact cells in the retentate to form a lysed/disrupted cell mixture comprising nucleic acid from lysed/disrupted cells; filtering the lysed/disrupted cell mixture through the filter to form a filtrate comprising nucleic acid from the lysed/disrupted cells; and detecting the presence of a mammalian housekeeping gene such as GAPDH DNA or a fragment thereof in the filtrate and estimating the number of UTC (eUTC) in the sample. In some aspects, the method further comprises classifying the subject as responsive to the administered kidney cancer treatment, or as non-responsive to the administered kidney cancer treatment, based on the quantification of the target analyte, amount of UTC or tumor cell density (eUTC), compared before, during and after administration of the cancer treatment. For e.g., if the quantity of the target analyte, amount of UTC or tumor cell density (eUTC) is lower during and/or after administration of kidney cancer treatment compared to the quantity of UTC or UTC density (eUTC) before treatment, then the subject can be classified as responsive to the administered cancer treatment with respect to tumors in the kidney and related urinary tract. In such aspects, the method can further comprise modifying the kidney cancer treatment, and further monitoring of the subject using the method disclosed herein.

In some aspects, the disclosure provides a method for evaluating the therapeutic efficacy of a treatment for a kidney cancer. In such aspects, the method comprises obtaining urine sample from the subject, wherein the urine samples are obtained before, during and/or after administration of a kidney cancer treatment. The samples obtained are then passed through the filtration member comprising a membrane having a pore size of about 8 microns to obtain a retentate, wherein the retentate is enriched in UTC compared to the urine sample; lysing or disrupting the intact cells in the retentate to form a lysed/disrupted cell mixture comprising nucleic acid from lysed/disrupted cells; filtering the lysed/disrupted cell mixture through the filter to form a filtrate comprising nucleic acid from the lysed/disrupted cells; and detecting the presence of a mammalian housekeeping gene such as GAPDH DNA or a fragment thereof in the filtrate, and estimating the number or concentration of UTC (eUTC) in the urine sample. In some aspects, the method further comprises classifying the kidney cancer treatment as effective, or as non-effective, based on the quantification of the target analyte, amount of UTC or tumor cell density, compared before, during and after administration of the cancer treatment. For e.g., if the quantity of the target analyte, amount of UTCs or tumor cell density (eUTC) is lower during and/or after administration of kidney cancer treatment compared to the quantity of the target analyte, amount of UTCs or tumor cell density before treatment, then the treatment can be classified as effective with respect to tumors in the kidney and related urinary tract. In such aspects, the method can further comprise modifying the kidney cancer treatment, and further evaluating the treatment using the method disclosed herein.

In further aspects, provided herein is a method for screening a composition for activity in treating a kidney cancer. In such aspects, the method comprises obtaining urine sample from the subject, wherein the urine samples are obtained before, during and/or after administration of a treatment with a candidate composition. The samples obtained are then passed through the filtration member comprising a membrane having a pore size of about 8 microns to obtain a retentate, wherein the retentate is enriched in UTC compared to the urine sample; lysing or disrupting the intact cells in the retentate to form a lysed/disrupted cell mixture comprising nucleic acid from lysed/disrupted cells; filtering the lysed/disrupted cell mixture through the filter to form a filtrate comprising nucleic acid from the lysed/disrupted cells; and detecting the presence of a mammalian housekeeping gene such as GAPDH DNA or a fragment thereof in the filtrate. In some aspects, the method further comprises classifying the composition as effective, or as non-effective, based on the quantification of the target analyte, amount of UTC or tumor cell density (eUTC), compared before, during and after administration of the candidate composition. For e.g., if the quantity of the target analyte, amount of UTC or tumor cell density (eUTC) is lower during and/or after administration of candidate composition compared to the quantity of the target analyte, amount of UTC or tumor cell density (eUTC) before administration of the candidate composition, then the composition can be classified as effective. In some aspects, if the quantity of the target analyte, amount of UTC or tumor cell density (eUTC) is elevated during and/or after administration of candidate composition compared to the quantity of the target analyte, amount of UTC or tumor cell density (eUTC) before administration of the candidate composition, then the composition can be classified as non-effective.

In some aspects, the disclosure provides a method for staging the kidney cancer. In such aspects, the method comprises obtaining urine sample from the subject, wherein the urine samples are obtained from a subject suspected of having kidney cancer. In some aspects, the subject is diagnosed as having kidney cancer and have been administered a cancer treatment. In such aspects, the urine samples can be obtained before, during and/or after administration of a cancer treatment. The samples obtained are then passed through the filtration member comprising a membrane having a pore size of about 8 microns to obtain a retentate, wherein the retentate is enriched in UTC compared to the urine sample; lysing or disrupting the intact cells in the retentate to form a lysed/disrupted cell mixture comprising nucleic acid from lysed/disrupted cells; filtering the lysed/disrupted cell mixture through the filter to form a filtrate comprising nucleic acid from the lysed/disrupted cells; and detecting the presence of a mammalian housekeeping gene such as GAPDH DNA or a fragment thereof in the filtrate. In some aspects, the method further comprises staging the cancer, based on the quantification of the target analyte, amount of UTC or tumor cell density (eUTC) within the urine sample, compared to a reference amount in for e.g., a kidney cancer-positive and/or kidney cancer-negative reference levels. In some aspects, the reference amount amounts from various stages including lower and higher stages of previously diagnosed kidney cancer. For e.g., if the quantity of the target analyte, amount of UTC or tumor cell density (eUTC) is higher. In some aspects, the method can further comprise administration of a cancer treatment and/or modifying the cancer treatment, and further evaluating the treatment using the method disclosed herein. In some aspects, distinguishing the stage of cancer may be valuable information in determining a course of treatment.

In some aspects, the disclosed herein is a method for monitoring progression/regression of kidney cancer in a subject. In such aspects, the method comprises obtaining or having obtained the urine sample from the subject, wherein the urine sample is obtained at a first time point and at least a second time point. The method further comprises passing the urine sample through a filtration member comprising a membrane having a pore size of about 8 microns to obtain a retentate, wherein the retentate is enriched in kidney tumor cells compared to the urine sample; lysing or disrupting the intact cells in the retentate to form a lysed/disrupted cell mixture comprising nucleic acid from lysed/disrupted cells; filtering the lysed/disrupted cell mixture from (c) through the filter to form a filtrate comprising nucleic acid from the lysed/disrupted cells; detecting the presence of a mammalian housekeeping gene such as GAPDH DNA or a fragment thereof in the filtrate. In such aspects, the quantity of the target analyte, amount of UTC or tumor cell density (eUTC) is compared to the quantity of the target analyte, amount of UTC or tumor cell density (eUTC) obtained from the subject at different time points. In such aspects, if the quantity of the target analyte, amount of UTC or tumor cell density (eUTC) is elevated in the second point of time compared to the quantity of the target analyte, amount of UTC or tumor cell density (eUTC) from the subject in the first point of time, it can indicate that the cancer in the subject has progressed. In some aspects, if the quantity of the target analyte, amount of UTC or tumor cell density (eUTC) is lower in the second point of time compared to the quantity of the target analyte, amount of UTC or tumor cell density (eUTC) from the subject in the first point of time, it can indicate that the cancer in the subject has regressed. In some aspects, distinguishing between types of cancer (e.g., less vs. more aggressive) may be valuable information in determining a course of treatment.

In some aspects, the GAPDH DNA can be detected and/or quantified using a probe comprising a nucleotide sequence set forth in SEQ ID NO: 1, forward primer comprising a nucleotide sequence set forth in SEQ ID NO: 2, reverse primer comprising nucleotide sequence set forth in SEQ ID NO: 3, or any combination thereof. In some aspects, the probe comprises a sequence with at least about 80% (e.g., about 85%, about 90%, about 95%, about 98%) identity to the nucleotide sequence of SEQ ID NO: 1. In some aspects, the forward primer comprises a sequence with at least about 80% (e.g., about 85%, about 90%, about 95%, about 98%) identity to the nucleotide sequence of SEQ ID NO: 2. In some aspects, the reverse primer comprises a sequence with at least about 80% (e.g., about 85%, about 90%, about 95%, about 98%) identity to the nucleotide sequence of SEQ ID NO: 3.

In some aspects, the methods disclosed herein may guide or assist in deciding a treatment path for a subject, for example, whether to implement procedures such as surgical procedures (e.g., full or partial nephrectomy), treat with drug therapy, or employ a watchful waiting approach.

In some aspects, the disclosed method and the device described herein can be applied to cancers that shed tumor cells into urine, for e.g., bladder cancer or other urogenital-related cancers. In such aspects, the cancer may be bladder cancer, urinary tract cancer, prostate cancer, or testicle cancer. In some aspects, the cancer cells isolated comprise bladder cancer cells, urinary tract cancer cells, prostate cancer cells, or testicle cancer cells. In some aspects, the cancer cells isolated using the device disclosed herein can further be used to detect a target analyte such as a housekeeping gene, or a discerning sequence.

In some aspects, the method comprises obtaining a sample from a subject at risk of having, suspected of having, or has a current or prior diagnosis of bladder cancer, urinary tract cancer, prostate cancer, or testicle cancer, passing the urine sample through a filtration member comprising a membrane having a pore size of about 8 microns to obtain a retentate, wherein the retentate is enriched in tumor cells compared to the urine sample; lysing or disrupting the intact cells in the retentate to form a lysed/disrupted cell mixture comprising nucleic acid from lysed/disrupted cells; filtering the lysed/disrupted cell mixture from (c) through the filter to form a filtrate comprising nucleic acid from the lysed/disrupted cells; detecting the presence of a mammalian housekeeping gene such as GAPDH DNA or a fragment thereof in the filtrate. In such aspects, the quantity of the target analyte, amount of UTC or tumor cell density (eUTC) is compared to the quantity of the target analyte, amount of UTC or tumor cell density (eUTC) obtained from a reference sample. In such aspects, the subject at risk or suspected of having the cancer may have elevated level of GAPDH DNA or a fragment thereof compared to the reference level. In some aspects, the subject at risk or suspected of having kidney cancer may have elevated level of UTC or tumor cell density (eUTC) as compared to the reference level. In such aspects, the reference level may be level of or average level of the target analyte, amount of cells or cell density in the urine sample of a subject or subjects without cancer.

### III. Processed Test Samples

A further aspect of the present disclosure provides processed test samples prepared from an original test sample for PCR amplification of nucleic acid from UTC suspected of being present in the original test sample. The processed test sample herein comprises the nucleic acid or protein extracts from cells and is substantially free of both free microbial nucleic acid, nucleic acid from blood cells and human cells present in the original sample.

The processed test sample can be prepared using any methods described above, starting from an original test sample described above, suspected of containing UTC, free nucleic acids from blood cells and microbial cells. In various aspects, the processed test sample is obtained by a process comprising a first filtering of the original test sample through a filter to form a retentate, wherein the retentate comprises a majority of the UTC from the test sample with almost undetectable level of blood and/or microbial cells; lysing the cells in the retentate to form a lysed cell mixture comprising nucleic acid from the lysed cells; and filtering the lysed cell mixture through the filter to form a filtrate comprising the nucleic acid from the lysed cells. In various aspects, the processed test sample is free of nucleic acid from dead microbial cells and blood cells.

In some aspects, lysing the cells comprises adding a lysis solution to the retentate suspected of comprising UTC. Any of the lysis solutions known in the art may be used for this purpose. In some aspects, the lysis solution comprises one or more of buffers, detergents, salts, or DNAse inhibitors, protease inhibitor, RNAase inhibitor. In various aspects, lysing the cells comprises agitating the UTC in the retentate in the presence of beads, granules, pellets or other solid material suitable for disrupting cell membranes. Any bead available to facilitate the lysis of cells can be used. For example, lysing the cells can comprise using glass beads, chrome beads, steel beads, chrome-steel beads, silicon carbide beads, garnet beads, stainless steel beads, granules, pellets, plastic beads, ceramic beads, or a combination of any thereof. The agitation can comprise mechanical agitation methods including, but not limited to, sonication, magnetic, forced gas, etc. In various aspects, the beads are glass beads. The beads can be of any size conventionally used in the art. For example, the beads can be from about 0.5 to 5 mm. In various aspects, the filter used to obtain the processed test sample does not bind nucleic acids or poorly binds nucleic acids such that a filtrate formed from filtering the original test sample through the filter comprises a majority of free nucleic acid present in the original test sample.

In various aspects, the processed test sample is prepared from a process further comprising rinsing the filter, filtrate, and/or retentate with a suitable non-DNA binding solution (e.g., water). In some aspects, the processed test sample comprises cell extracts, for example protein extracts from UTC present in the original sample.

### IV. Kits

A further aspect of the present disclosure provides kits (for example Filter PCR) comprising the device as provided herein and instruction for use. In various aspects, the kits can be prepared to identify, quantify, sequence or otherwise detect target analytes related to kidney cancer from urine samples. In various aspects, the kit can be prepared to identify, quantify, sequence or otherwise detect target nucleic acid from example GAPDH from UTC in urine samples. In various aspects, the kits can be prepared to identify, quantify, sequence or otherwise detect protein or peptides from UTC in urine samples. Accordingly, the kits can be formulated with reagents and materials useful for performing any of the steps described herein. The reagents may include but are not restricted to cell lysis buffer, SDS, DNA isolation reagents, DNA precipitation reagents, RNA isolation reagents, protein precipitation reagents, protein purification reagents, antibodies, enzymes, dyes, organic and inorganic solutions, buffers, suspensions etc. In further aspects, the kit can further comprise beads, granules, pellets, or other solid materials.

In some aspects the kit (Filter PCR) may comprise reagents (e.g., primers, probes, buffers) for conducting a polymerase chain reaction to amplify a nucleic acid of the intact cell of interest (e.g. UTCs). In various aspects, the kit can comprise reagents for analyzing an amplified nucleic acid of the intact cell. These can include, for example, standard control nucleic acids (e.g., DNAs) for DNA quantification purposes. Accordingly, the kits can further comprise a buffer, a nucleic acid probe, an oligonucleotide primer, barcoding solutions, an enzyme, a substrate, a gel, a nucleic acid dye, a microarray, a control nucleic acid, or a combination of any thereof. In some particular aspects, the kit may comprise suitable primers to amplify a region of the GAPDH gene.

The kits provided herein generally include instructions for carrying out the methods detailed herein. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

Detection of UTC in cancer patients during early phases of the disease has so far been a challenge. The current state-of-the-art uses microscopic analysis of urine samples, which is prone to very high false negatives. Though multiple biomarkers are associated with kidney cancer, the testing procedure is complicated. The current disclosure provides a novel device and methods that can isolate kidney tumor cells from urine, which can then be accurately quantified and compared with self or non-self-samples to make a diagnosing and staging kidney cancer.

### Example 1. Capture of Human Kidney Cancer Cells by Filtration

Study was conducted to test that the filter system can effectively capture whole Renal Cell Carcinoma (RCC) cells from live RCC culture samples.

### Materials and Methods

Samples: 10 ml of human RCC cell suspension was obtained from a RCC culture and was divided into two 50 ml tubes (˜5 ml each) including a filter tube (RCC-F) and a non-filter tube (RCC-C).

Filter. 8-micron filter custom made for removing blood cells and which can hold up to 20 ml liquid was used.

The RCC-F tube (see Table 1) containing ˜5 ml RCC culture suspension was centrifuged at 1000 rpm for 1 minute and the filter was transferred into a new 50 ml tube (RCC-F), saving the filtrate (RCC-A) for later DNA isolation. 1 ml of SDS extraction buffer was added onto the filter of RCC-F. Glass beads for cell disruption were added onto the filter. The sample was then shaken in a laboratory shaker for 3 minutes and centrifuged at 4000 rpm for 5 minutes. The filter was subsequently discarded and the filtrate saved and transferred into a 2 ml tube. The sample was then treated with 5M potassium acetate, mixed on a shaker and centrifuged. The supernatant was then transferred into new tubes containing equal volume of isopropanol. The samples were vortexed followed by centrifugation. The supernatant was discarded and the pellet treated with 70% ethanol and then centrifuged. The precipitate was then dried and resuspended in 0.1×TE buffer.

The RCC-A containing 5 ml filtrate from RCC-F and RCC-C containing 5 ml sample (see Table 1A) were centrifuged at 4000 rpm for 5 minutes and the precipitate from each sample was treated with SDS extraction buffer, and the resuspension was transferred into 2 ml tubes containing glass beads for cell disruption. The samples were then subjected to shaking on a shaker for 3 minutes and then centrifuged. The samples were then treated with 5M potassium acetate and mixed at a shaker followed by centrifugation. The resulting supernatant was transferred into a new microfuge tube and treated with isopropanol, vortexed and centrifuged. The samples were then treated with 70% ethanol and centrifuged. The precipitate was dried and resuspended in 0.1×TE buffer.

5 μl of each sample was used for qPCR analysis for detection and quantification of the human GAPDH gene, probe and primers provided in Table 1B. The gene copy number of GAPDH from these three samples were compared.

TABLE 1A
Description of Test System

| Sample | Description |
|---|---|
| RCC-F | RCC sample in container with a filter, or comprised of a filter |
| | tube |
| RCC-A | The filtrate from RCC-F or a container containing the same |
| | filtrate |
| RCC-C | RCC sample in container without a filter or a filter tube |

TABLE 1B
Probe and primers used in the qPCR

| | Seq ID | |
|---|---|---|
| | NO. | Sequence |
| Probe | 1 | AGCCACACCATCCTAGTTGCC |
| Forward primer | 2 | GGAAGACAGAATGGAAGA |
| Reverse primer | 3 | CAAGGTTACCATATACCCA |

Table 2 shows that the filter was able to capture about 1.18×10⁴ intact RCC cells and about 6.29×10⁵ dead RCC cells and cell free DNA copies passed through the filter due to potential damaged cells or dead cells in the process. The total cell count and DNA copy including intact and dead cells and cell-free DNA was estimated to be 1.32×10⁶ per Test sample. Further, copy estimates relates to the cell number in the sample. The results of this study demonstrated that the filter captured intact RCC cells and in sufficient quantity for accurate PCR and quantitative PCR analysis.

TABLE 2
Cell DNA Copy Number in Different Treatments

| Sample | Copy/μl in DNA | Copy in 5 ml | Comments |
|---|---|---|---|
| RCC-F | 236.71 | 1.18E+04 | Intact RCC on filter |
| RCC-A | 12570.696 | 6.29E+05 | Dead RCC/cfDNA in filtrate |
| RCC-C | 26381.727 | 1.32E+06 | Total cells (intact + dead) + |
| | | | cfDNA* |

Note:
the copy estimate could be equivalent to cell number
*cell free DNA

The data from this study further confirmed that a GAPDH-specific assay can detect RCC as GAPDH exists in all tumor cells with/without known mutations.

### Example 2. Separation of Mammalian Blood Cells Through the Filter

Study was conducted to evaluate the efficiency of the filter in filtering, removing, and/or separating mammalian (for e.g., human) blood cells from other materials in a solution. The presence of blood in urine (hematuria) is a common symptom of renal cancer once the disease has progressed to middle or advanced stages. Additionally, blood in urine can be commonly present in subjects with Urinary tract infections (UTIs). If blood cells are present in a urine sample, that could be a contaminating factor for detection of kidney cancer cells by DNA in urine. If a filtration method can capture intact renal cancer cells and separate cancer cells from blood cells and cell-free DNA, then the process is capable of utilizing the DNA present in the retained cancer cells such as RCC for use in a diagnostic screen for identification and quantification of the cancer cells.

A sample was generated using diluted whole human blood added to human urine. The sample was filtered with an 8-micron filter as described below and DNA was extracted from the retentate on the filter and the filtrate that passed though the filter. These were subjected to PCR analysis. Amplification for the presence and quantity of a sequence specific to the human GAPDH gene was used to establish copy number per μl from these two DNA samples, which were compared to evaluate the filter efficiency in removing blood cells from a sample.

### Materials and Methods

Samples: 1/100 diluted whole human blood sample purchased from a vendor; urine sample from a person that tested negative by CT scan for kidney cancer.

Filter. 8-micron filter custom made for removing blood cells and can hold up to 20 ml liquid was used.

DNA extraction: One ml of 1/100 diluted whole blood was added into 48 ml urine sample (tested negative for kidney cancer). 17 ml of the mixed sample was then added onto the filter (as described in example 1) within a 50 ml tube (UBF) and was then centrifuged at 1000 rpm for 1 minute. After centrifugation, the filtrate was transferred to a new tube (to create sample UBA). The process was repeated until all 49 ml of sample was filtered, and 49 ml filtrate was transferred into a fresh tube labeled UBA for later DNA extraction. The filter was then transferred into a new 50 ml tube (UBF) and treated with SDS extraction buffer along with glass beads for cell disruption. The sample was then shaken in a laboratory shaker for 3 minutes and centrifuged at 4000 rpm for 5 minutes. The filter was subsequently discarded and the filtrate saved and transferred into a 2 ml tube. The sample was then treated with 5M potassium acetate, mixed on a shaker and centrifuged. The supernatant was then transferred into new tubes containing equal volume of isopropanol. The samples were Vortexed followed by centrifugation. The supernatant was discarded and the pellet treated with 70% ethanol and then centrifuged. The precipitate was then dried and resuspended in 0.1×TE buffer.

The UBA containing 49 ml filtrate from UBF was centrifuged at 4000 rpm for 5 minutes and the precipitate was treated with SDS extraction buffer, and the resuspension was transferred into 2 ml tubes containing glass beads for cell disruption. The samples were then subjected to shaking on a shaker for 3 minutes and then centrifuged. The samples were then treated with 5M potassium acetate and mixed at a shaker followed by centrifugation. The resulting supernatant was transferred into a new microfuge tube and treated with isopropanol, Vortexed and centrifuged. The samples were then treated with 70% ethanol and centrifuged. The precipitate was dried and resuspended in 0.1×TE buffer.

5 μl of each sample was used for qPCR analysis for presence and quantity of the human GAPDH gene. The gene copy number of GAPDH from these three samples were compared.

Table 3 shows that no DNA was detected from the retentate (UBF) on the filter. An estimated 1.1×10⁶ copies of GAPDH (or blood cell number) were found from the filtrate (UBA). The results demonstrated that the filter can effectively pass-through blood cells and its DNA. Removal of blood cells and cell free DNA helps to increase the accuracy for the detection method for renal tumor cells that are retained on or by the filter.

TABLE 3
Cell DNA Copy Number in Different Treatments

| Sample | Copy/μl | Copy in 49 ml | Comments |
|---|---|---|---|
| UBA | 22.135 | 1106.75 | Blood cells and call-free |
| | | | DNA in filtrate |
| UBF | 0 | 0 | Blood cells on filter |

### Example 3. Detection of RCC in Test Urine Samples Mixed with RCC on the Filter

In Examples 1-2, the filter was demonstrated to capture RCC cells and to remove blood cells. Study was further conducted to test using the filter to capture RCC cells from a urine sample mixed with RCC. This is significant because there are no prior examples of RCC capture in urine and/or utilization of RCC cells for the purposes of cancer cell DNA analysis and diagnosis. The ability to effectively analyze diagnostic DNA from urine has been limited due to several factors, including low pH. Extraction and detection of specific DNA fragments from captured intact RCC cells in urine is diagnostic for presence of cancer cells (for e.g., RCC) within the urinary tract. Likewise, elimination of blood cells and contaminating cell free human DNA from the urine sample by specific filtration allows use of a general housekeeping gene DNA sequence (e.g., GAPDH) as a definitive screening tool for suspicion of kidney cancers. A quantitative PCR detection system based on a ubiquitous yet essential, so-called human housekeeping gene, has an added advantage of being unlikely to contain significant mutations which enables detection of cancers harboring a wide variety of genetic sequence defects, as is seen in renal cancers. Detection of a human-specific DNA sequence also eliminates potential false positive results from spurious DNA derived from microorganisms or minor presence of healthy human cell contaminants whose presence falls below the significance threshold of the assay and/or are removed from the final test sample (blood cells being one example).

### Materials and Methods

Samples: 3 ml RCC cell suspension was obtained from a live RCC culture. 1 ml was added into each of two 50 ml tubes containing 48 ml of healthy urine (sample source confirmed negative for kidney cancer). One tube of mixed samples was subjected filtration (as described in the previous examples) and labeled as URF. The other tube was not treated with the filter and labeled as URC.

Filter. 8-micron filter custom made for removing blood cells and can hold up to 20 ml liquid was used.

17 ml of the mixed sample (URF) was added onto the filter within a 50 ml tube (URF) and was then centrifuged at 1000 rpm for 1 minute. After centrifugation, the filtrate was transferred to a new tube (URA). The process of filtration and centrifugation was repeated until 49 ml of sample was filtered and 49 ml filtrate was transferred into tube URA for later DNA extraction. The filter was then transferred into a new 50 ml tube (URF) and treated with SDS extraction buffer along with glass beads for cell disruption. The sample was then shaken in a laboratory shaker for 3 minutes and centrifuged at 4000 rpm for 5 minutes. The filter was subsequently discarded and the filtrate saved and transferred into a 2 ml tube. The sample was then treated with 5M potassium acetate, mixed on a shaker and centrifuged. The supernatant was then transferred into new tubes containing equal volume of isopropanol. The samples were vortexed followed by centrifugation. The supernatant was discarded and the pellet treated with 70% ethanol and then centrifuged. The precipitate was then dried and resuspended in 0.1×TE buffer.

DNA extraction: The URA containing 49 ml filtrate from URF and URC containing 49 ml sample were centrifuged at 4000 rpm for 5 minutes and the precipitate from each sample was treated with SDS extraction buffer, and the resuspension was transferred into 2 ml tubes containing glass beads for cell disruption. The samples were then subjected to shaking on a shaker for 3 minutes and then centrifuged. The samples were then treated with 5M potassium acetate and mixed at a shaker followed by centrifugation. The resulting supernatant was transferred into a new microfuge tube and treated with isopropanol, vortexed and centrifuged. The samples were then treated with 70% ethanol and centrifuged. The precipitate was dried and resuspended in 0.1×TE buffer.

5 μl of each sample was used for qPCR analysis for presence and quantity of the human GAPDH gene. The gene copy number of GAPDH from these three samples were compared.

URF data showed that the filter was able to capture about 6652 intact RCC cells and URA data showed about 6093.35 dead RCC cells and other copies of free human DNA passed through the filter from to potential damaged cells, dead cells, and/or blood cells and related human DNA in the process (Table 4). URC showed the total cell count and DNA copy including live and dead cells and cell-free DNA and were estimated to be 6.68×10⁴.

In addition, this study further proved that GAPDH assay can be used to detect tumor cells such as RCC and eliminated a major contaminating source of human DNA from normal cells in urine.

TABLE 4
Comparison of Copy Number from Different Treatments

| | Copy/μl | | |
|---|---|---|---|
| Sample | in DNA | Copy in 49 ml | Comments |
| URF | 133.044 | 6652.2 | Intact RCC on filter |
| URA | 120.867 | 6043.35 | Dead RCC and cfDNA |
| | | | in filtrate |
| URC | 1335.573 | 66778.65 | Total cells (intact + |
| | | | dead) + cfDNA* |

*Cell-free DNA in the sample

### Example 4. Detection of Kidney Tumor Cells in Urine Samples of a Kidney Cancer Patient

The following analysis was performed to test whether a PCR assay targeting a housekeeping gene (GAPDH) can be used to detect tumor cells in urine from a kidney cancer patient at early stage prior to development of symptoms. A 62-year-old, male patient at stage 1 asymptomatic kidney cancer (urinalysis showed no hematuria) was diagnosed with kidney cancer after a CT-scan for unrelated issues. A CT-scan showed two lesions (one cystic and one solid) in his left kidney. After the nephrectomy, the pathologist was able to identify the larger neoplasm as a 3.8 cm ccRCC tumor, pT1a, ISUP grade 3. The second 2.3 cm renal cell neoplasm was identified as an oncocytoma. With the patient's consent, 50 mL of urine sample was collected 2 days before nephrectomy, 14 days after nephrectomy and 60 days after nephrectomy.

The cells from urine samples were retained after centrifugation as precipitate at the bottom of the 50 ml tube. Only the sample from before nephrectomy showed visible cell precipitate at the bottom of the 50 ml tube after the centrifugation and other samples from post-nephrectomy did not show visible precipitate. Lysis buffer A and glass beads were added into the precipitate to mix and then release DNA in supernatant. The supernatant was treated with 5 M potassium acetate and then centrifuged. The supernatant was then transferred to mix with equal amount of isopropanol to precipitate the DNA by centrifugation. 70% ethanol was then added to the precipitate to purify the DNA by centrifugation. The precipitate was let dry and then resuspended with 0.1×TE buffer as the final DNA product. The isolated DNA product was then used for qPCR analysis for GAPDH using standard primers and probe that exists in all tumor cells with/without known mutations.

Data suggests that CTC can not only be isolated from urine of patients, but accurately quantified using qPCR analysis for housekeeping genes, for example GAPDH. The gene copy/ml obtained from qPCR was then used to estimate the CTC density per ml in urine (Table 5).

TABLE 5
Cell density among samples

| | cells/ml | log cells/ml |
|---|---|---|
| Before Surgery | 459 | 2.66 |
| 2 weeks post-surgery | 3.78 | 0.58 |
| 2 months post-surgery | 0 | 0 |

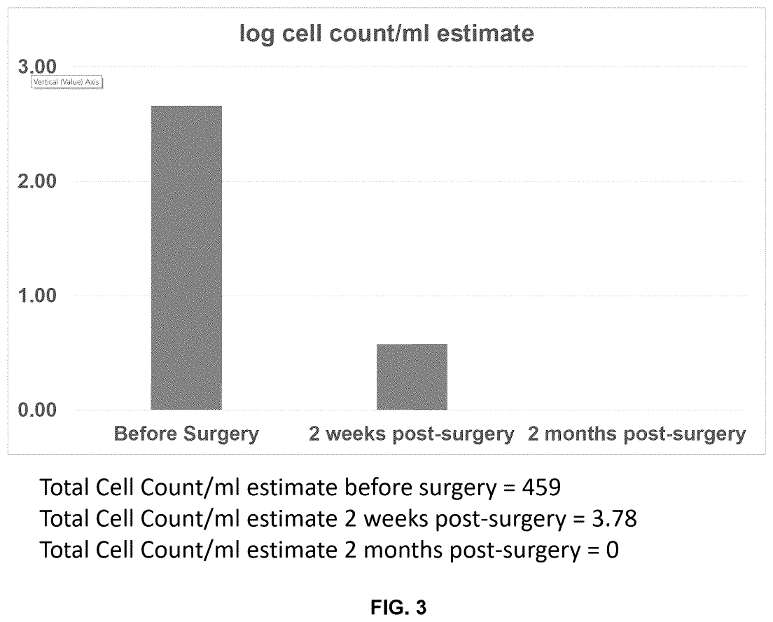

This information provides more accurate information than the standard urinalysis which is much less sensitive due to the limited amount of urine (a drop) is used for microscopic analysis. The results showed that the CTC density was estimated to be 459 cells/ml before nephrectomy, 3.78 cells/ml two weeks after nephrectomy, and 0 cells/ml two months after nephrectomy (FIG. 3).

### Example 5. Detection of Kidney Tumor Cells in Urine Samples of Ten Kidney Cancer Patients

In the previous examples, the custom filter was shown to capture RCC cells and remove blood cells and cell-free DNA when RCC was mixed with urine samples. In this study, urine samples from ten patients who were at early stage of kidney cancer and who had no symptoms for kidney cancer were used to test the detection method with the disclosed custom filter. The study was conducted to ensure that the tumor cells can be enrich and captured on the custom filter and tumor-specific DNA can be isolated from patients' urine and quantified with our validated qPCR assay that is GAPDH-specific. The data from this study can help us to optimize our testing process and also serve as a proof of concept for our technology.

### Materials and Methods

Samples: With appropriate clinical trial permit, urine samples from ten verified Renal Cancer patients were taken prior to surgery to remove the renal tumor(s). We were informed that these patients had no cancer symptoms and were diagnosed to be positive for kidney cancer incidentally by CT-scan. 50 to 100 ml of urine samples were taken from these patients. 50 ml urine sample from each patient was used for filter treatment (UF) and the remaining urine sample was then used for non-filter treatment (UC).

Filter. 8-micron filter custom made for removing blood cells.

DNA extraction: DNA extraction was conducted soon after a urine sample was received. 17 ml of the urine sample was added onto the filter within a 50 ml tube (UF) and was then centrifuged at 1000 rpm for 1 minute. After centrifugation, the filtrate was transferred to a new tube (UA). Kept repeating this process until 50 ml of sample was filtered and total 50 ml filtrate was transferred into tube UA for later DNA extraction. The filter was then transferred into a new 50 ml tube (UF) and treated with SDS extraction buffer along with glass beads for cell disruption. The sample was then shaken in a laboratory shaker for 3 minutes and centrifuged at 4000 rpm for 5 minutes. The filter was subsequently discarded, and the filtrate saved and transferred into a 2 ml tube. The sample was then treated with 5M potassium acetate, mixed on a shaker and centrifuged. The supernatant was then transferred into new tubes containing equal volume of isopropanol. The samples were vortexed followed by centrifugation. The supernatant was discarded and the pellet treated with 70% ethanol and then centrifuged. The precipitate was then dried and resuspended in 0.1×TE buffer.

The UA containing 50 ml filtrate from UF and UC containing 50 ml sample were centrifuged at 4000 rpm for 5 minutes and the precipitate from each sample was treated with SDS extraction buffer, and the resuspension was transferred into 2 ml tubes containing glass beads for cell disruption. The samples were then subjected to shaking on a shaker for 3 minutes and then centrifuged. The samples were then treated with 5M potassium acetate and mixed at a shaker followed by centrifugation. The resulting supernatant was transferred into a new microfuge tube and treated with isopropanol, vortexed and centrifuged. The samples were then treated with 70% ethanol and centrifuged. The precipitate was dried and resuspended in 0.1×TE buffer.

5 μl of each sample was used for qPCR analysis for presence and quantity of the human GAPDH gene. The gene copy number of GAPDH from these three samples were compared.

Table 6 displays the differences among the different sample fractions from the same patient. Each urine sample is expected to contain tumor cells, blood cells, and cell-free DNAs. The tumor cell number may vary based on person and the disease status such as the location of the tumor(s) and the pathology grade classification of the tumor cells. The tumor cell number correspondent to copy number from DNA isolated from the filter. Therefore, the copy number for UF can represent the disease status of kidney cancer. In fact, the copy number for UF is the novel biomarker we propose. With the disclosed custom filter and GAPDH assay, the tumor cells was detected and quantified (see sample UF of Table 6). This shows that the disclosed method and device provides a low-risk quick screening process for renal cancer, suitable as an initial diagnostic tool or part of a routine annual screen for patients in certain risk categories.

TABLE 6
Gene Copy Number from Urine Samples
of Ten Renal Cancer Patients

| | Tumor | | | | |
|---|---|---|---|---|---|
| Patient | size (cm) | UF | UA | UC | UC − UA |
| 1 | 6.5 | 3.55E+03 | 1.22E+03 | 1.77E+04 | 1.65E+04 |
| 2 | 8.8 | 1.04E+05 | 1.12E+06 | 1.90E+06 | 7.89E+05 |
| 3 | 2.2 | 1.80E+04 | 1.88E+04 | 3.56E+05 | 3.37E+05 |
| 4 | 3.9 | 2.58E+03 | 1.81E+03 | 2.31E+03 | 5.04E+02 |
| 5 | 1.9 | 3.17E+02 | 1.06E+02 | 2.15E+02 | 1.09E+02 |
| 6 | 3.3 | 2.50E+03 | 1.90E+04 | 8.63E+04 | 6.73E+04 |
| 7 | 3.4 | 7.32E+03 | 2.58E+04 | 3.50E+04 | 9.18E+03 |
| 8 | 3 | 1.06E+03 | 1.03E+05 | 2.98E+05 | 1.95E+05 |
| 10 | 2.4 | 7.57E+03 | 6.81E+03 | 1.85E+04 | 1.17E+04 |

UF: DNA isolated from captured tumor cells on the filter;
UA: DNA isolated from the filtrate that passed through the filter (e.g., derived from dead cells, bloods cells, other free human DNA);
UC: DNA isolated from samples without filter treatment.
Gene (sequence) copy number was estimated based on a 50 ml urine sample.
UC − UA: Data from UA was subtracted from data from UC to compare with UF.

When the experiment was designed, UC was the sample without treatment and used as a control to obtain the total DNA. The sample treated with filter produced two data including data from the filter (UF) and data from the filtrate (UA). As a result, it was expected that UF should be closely equivalent to the difference between UC and UA. The log value for UF and UC-UA were compared and the average value from these two data from ten samples was also obtained (Table 7). The relative standard deviation in percentage was then calculated using this formula:

$$RSD = [ \frac{( mean UF - mean UC - UA )}{\frac{( mean UF + mean UC - UA )}{2}} ] \times 100$$

Using the formula, an RSD of 16.15% was obtained which met the acceptance criterion: <25%. This demonstrated that the filter was able to enrich the expected amount of tumor cells and produce accurate results for kidney cancer detection.

TABLE 7
Log Value Comparison to Evaluate Test Efficiency

| Patient | Log UF | Log UC − UA |
|---|---|---|
| 1 | 3.55 | 4.22 |
| 2 | 5.02 | 5.90 |
| 3 | 4.26 | 5.53 |
| 4 | 3.41 | 2.70 |
| 5 | 2.50 | 2.04 |
| 6 | 3.40 | 4.83 |
| 7 | 3.86 | 3.96 |
| 8 | 3.03 | 5.29 |
| 9 | 1.97 | 2.47 |
| 10 | 3.88 | 4.07 |
| Mean | 3.49 | 4.10 |

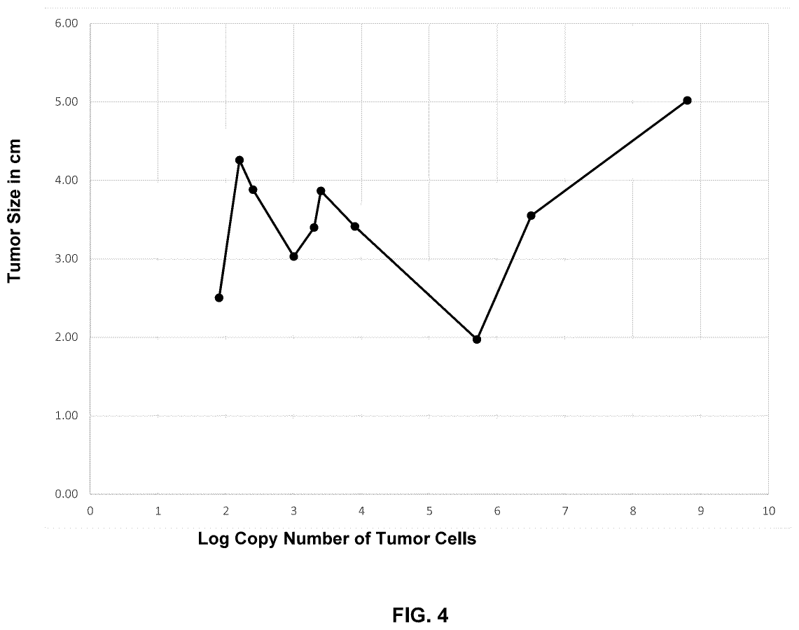

A trend between tumor cell size and tumor copy number was also observed (FIG. 4). Although it is not shown as a linear trend on the graph, the data is still very informative and insightful. As the samples were taken from patients at an early stage of kidney cancer (smaller tumors localized within the kidney interior). This may contribute to lower copy number of tumor cells for some patients. Further, the detection method was able to enrich the tumor cells and enable the production of tumor-cell-specific DNAs and estimate the amount of tumor cells. This will help to provide better and more accurate information on disease status of kidney cancer.

A detection method from urine, that is based on the tumor cell size difference to enrich kidney tumor cells for DNA isolation, has not been reported or used for kidney cancer detection in a clinical lab. Most kidney cancer detection methods test DNA from blood and a few of them may test DNA (total DNA) from urine. The data disclosed herein showed that only a small portion of the total DNA in urine was from tumor cells. Based on this finding, analysis of the cell-free DNA in urine sample without using the disclosed filtration method, may miss analyzing the actual tumor DNA in the urine sample. This is particularly true for kidney cancer which can arise from a number of different genetic mutations, evading mutation-specific DNA analyses methods. This may produce false negative results. The detection method disclosed herein based on isolating whole cancer cells from other contaminants in urine and utilizing a human-specific ‘housekeeping gene’ sequence separated from specific cancer mutations, can prevent occurrence of false negatives. GAPDH, a common housekeeping gene has thus been proven a valuable gene for kidney cancer detection in the disclosed study.

### Example 6. Detection of Kidney Tumor Cells in Urine Samples of a Kidney Cancer Patient

Further tests were conducted to evaluate the efficiency of the disclosed detection method for kidney cancer monitoring. Multiple tests were conducted and summarized from one patient. This study helped define the specificity of the detection method and whether the method of detection can be used to detect the residency of renal cancer cells in urine after diseased kidney removal.

Samples: A patient positive for RCC by CT-scan and by cell pathology examination, underwent a radical nephrectomy to remove the diseased kidney. The patient had no visible symptoms and only trace hematuria. 50 ml urine from the same patient was collected at four different times: (1) before surgery with tumor, (2) two weeks after surgery (3), two months after surgery, and (4) approximately one year after surgery. Samples derived from these urine collections were labeled as 7B, 516-3, F and AB-F, respectively. The samples were collected in a nonclinical setting with appropriate written consent of the patient with sample source and date verification.

Filters: Before surgery with the subject had ˜4 cm kidney tumor and a 0.2-micron filter tube was used to isolate DNA from urine sample (7B). 2-weeks post-surgery a 0.2-micron filter tube was used to isolate DNA from urine sample (516-3). 2-months after surgery, a 8-micron filter (pluriSelect) was used to isolate DNA from urine sample (F). 1 year after surgery, a the custom 8-micron filter was used to isolate DNA from urine sample (AB-F).

DNA extraction for sample 7B, 516-3 and F: 50 ml urine was centrifuged at 4000 rpm for 10 minutes to collect the precipitate. The precipitate was resuspended with SDS extraction buffer and transferred into a filter tube with 0.2-micro membrane or a commercial 8-micron filter (by pluriSelect) inserted on top of 15 ml tube. An appropriate DNA extraction procedure was then followed and isolate DNA from these samples.

DNA extraction for AB-F: This procedure was similar to the described procedure in previous examples.

5 μl of each sample was independently used for qPCR analysis for presence and quantity of a sequence specific to the human GAPDH gene. The gene copy/ml and copy of GAPDH from these two samples were compared.

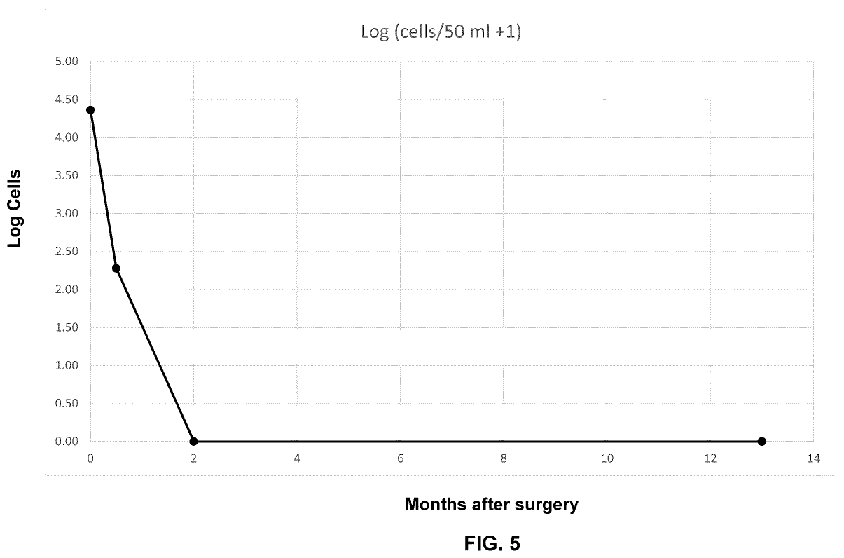

FIG. 5 and Table 8 demonstrated that the renal cancer tumor was generating a significant amount of free intact cancer cells in the patient urine before surgery (0 month). The number of cancer cells in urine dropped off precipitously after surgery to an undetectable level after 2 months post nephrectomy. This also confirmed that the detection assay to be effective in detecting tumor cell count through the copy number of GAPDH. Further, the results also confirmed that the filter-PCR method with the custom filter worked effectively.

TABLE 8
Data Summary from Samples Collected at Different Times

| Sample | Month | cells/50 ml | Cells/50 ml + 1 | Log (cells/50 ml + 1) |
|---|---|---|---|---|
| 7B | 0 | 22950 | 22951 | 4.36 |
| 516-3 | 0.5 | 189 | 190 | 2.28 |
| F | 2 | 0 | 1 | 0.00 |
| AB-F | 13 | 0 | 1 | 0.00 |

### SUMMARY

In summary, a filter-based device and method based on detecting and quantifying target DNA for identifying Kidney cancer was developed and optimized. The method further comprises estimating the amount of UTC or tumor cell density (eUTC) using the gene copy/ml obtained from qPCR. The device and method were successful in isolating and quantifying GAPDH across different tumor cell densities. Further, the filter disclosed herein was effective in removing blood cell genomic DNA from the samples. The device and methods disclosed herein provide a rapid identification of kidney cancer in a non-invasive manner. Since, the methods disclosed herein use a housekeeping gene for detecting and quantification of kidney cancer cells in urine and are not limited to specific biomarkers for detecting kidney cancer, therefore can accurately detect a range of kidney cancers.

1. A method for detecting a target analyte in cancer cells in a urine sample, the method comprising:
(a) obtaining or having obtained the urine sample from a subject;
(b) passing the urine sample through a filtration member to obtain a retentate, wherein the retentate is enriched in cancer cells, and wherein the filtration member is disposed concentrically within a first container member of a device; the filtration member comprising a clog-resistant filter disposed therein, the filter having a pore size of about 8 to about 12 microns; and
(c) detecting the target analyte in the retentate;
wherein the cancer cells are selected from kidney cancer cells, bladder cancer cells, urinary tract cancer cells, prostate cancer cells, and testicular cancer cells.

2. The method of claim 1, wherein the method further comprises: (d) quantifying the target analyte of (c).

3. The method of claim 2, wherein the method of quantification comprises a method selected from quantitative Polymerase chain reaction (qPCR), Reverse-Transcription PCR (RT-PCR), Multiplex PCR, Nested PCR, High Fidelity PCR, Fast PCR, Hot Start PCR, Long-range PCR, Arbitrary Primed PCR, Digital PCR, Droplet Digital PCR (ddPCR), isothermal amplification PCR, and Endpoint PCR (qualitative PCR).

4. The method of claim 2, wherein the target analyte comprises a discerning nucleic acid sequence or a housekeeping gene or a fragment thereof.

5. The method of claim 4, wherein the housekeeping gene is GAPDH.

6. The method of claim 2 wherein the quantification of target analyte is used to: (a) estimate a tumor cell density in urine; (b) identify the tumor cells in urine; or (c) characterize the phenotype of the tumor cells.

7. The method of claim 1, further subjecting the retentate to disruption of the cancer cells in the retentate and centrifugation to produce a filtrate comprising DNA from the cancer cells.

8. The method of claim 1, wherein the filtration member comprises a membrane with a pore size of about 8 microns.

9. The method of claim 1, wherein the subject is at risk of having, suspected of having, undergoing screening, or has a current or prior diagnosis of kidney cancer, bladder cancer, urinary tract cancer, prostate cancer, or testicle cancer.

10. The method of claim 6, further comprising indicating the subject as having cancer when the level of the target analyte the retentate or the tumor cell density in the urine sample is elevated compared to a reference level.

11. A device of claim 1, the device comprising:
(a) the container member having a bottom surface having a center point; and
(b) the filtration member having a center point, the filtration member concentrically positioned adjacent to the container member.

12. A method of treating a subject suspected of, at risk of having, or undergoing screening for a cancer, comprising:
(a) obtaining or having obtained a urine sample from the subject;
(b) passing the urine sample through a filtration member to obtain a retentate, wherein the retentate is enriched in cancer cells; and wherein the filtration member comprises a clog-resistant filter disposed therein, the filter having a pore size of about 8 to about 12 microns;
(c) lysing or disrupting the cells in the retentate to form a lysed/disrupted cell mixture comprising nucleic acid from lysed/disrupted cells;
(d) filtering the lysed/disrupted cell mixture from (c) through the filtration membrane to form a filtrate comprising the nucleic acid from the lysed/disrupted cells;
(e) quantifying the level of a target analyte present in the retentate;
(f) comparing the level of the target analyte in the urine sample to a reference level of the target analyte in a control sample; determining the tumor cell density of the urine sample in the retentate; or characterizing the phenotype of the tumor cells;
(g) indicating the subject as having cancer when the level of the target analyte or the tumor cell density in the urine sample is elevated compared to the reference level; and
(h) administering a treatment to the subject identified as having cancer;
wherein the cancer is selected from kidney cancer, bladder cancer, urinary tract cancer, prostate cancer, and testicular cancer.

13. The method of claim 12, wherein the target analyte comprises a housekeeping gene or a fragment thereof.

14. The method of claim 13, wherein the target analyte is GAPDH DNA or a fragment thereof.

15. The method of claim 12, wherein the filtration member comprises a membrane with a pore size of about 8 microns.

16. The method of claim 12, wherein (c) or (d) comprises:
(a) isolating a nucleic acid from the cancer cells in the retentate; and
(b) conducting a qPCR analysis using primers and probe targeting a discerning nucleic acid sequence, sequences, or a fragment thereof.

17. The method of claim 12, wherein the filtration member is disposed concentrically within a container member of a device, the container member having a bottom surface having a center point; and the filtration member having a center point, the filtration member concentrically positioned adjacent to the container member.

18. The method of claim 12, further comprising subjecting the subject identified as having cancer to in-depth clinical workup or imaging prior to step (a) or subsequent to step (g).

19. The method of claim 12, wherein the treatment comprises administering the subject identified as having cancer a treatment selected from a chemotherapeutic agent, an immunomodulatory drug, an anti-inflammatory agent, an anti-angiogenic agent, a checkpoint inhibitor, an immunotherapy, ablation, radiation, and surgery.

20. The method of claim 12, wherein the method of (e) is selected from quantitative Polymerase chain reaction (qPCR), Reverse-Transcription PCR (RT-PCR), Multiplex PCR, Nested PCR, High Fidelity PCR, Fast PCR, Hot Start PCR, Long-range PCR, Arbitrary Primed PCR, Digital PCR, Droplet Digital PCR (ddPCR), isothermal amplification PCR, and Endpoint PCR (qualitative PCR).

21. A device for preparing a urine sample from a subject for analysis to detect kidney cancer cells in a urine sample, the device comprising:
(a) a container member having walls defining a bottom surface having a center point; and
(b) a filtration member disposed concentrically within the first container member; the filtration member comprising a clog-resistant filter disposed therein, the filter having a pore size of about 8 to about 12 microns; and
(c) a urine sample contained within the filtration member;
wherein the cancer cells are selected from kidney cancer cells, bladder cancer cells, urinary tract cancer cells, prostate cancer cells, or testicular cancer cells.
